(19) United States
(12) Patent Application Publication
(10) Pub. No.: US 2018/0208886 A1
(43) Pub. Date: Jul. 26, 2018

(54) PROTEIN-RICH BIOMASS OF THRAUSTOCHYTRIDS, CULTURING METHOD AND USES

(71) Applicants: FERMENTALG, Libourne (FR); ADISSEO FRANCE SAS, Antony (FR)

(72) Inventors: Olivier CAGNAC, Libourne (FR); Cyril ROLS, Marseille (FR); Julien PAGLIARDINI, Bordeaux (FR); Pierre CALLEJA, Saint Sulpice et Cameyrac (FR); Cécile GADY, Montluçon (FR); Sabrina VANDEPLAS, Terjat (FR)

(73) Assignees: FERMENTALG, Libourne (FR); ADISSEO FRANCE SAS, Antony (FR)

(21) Appl. No.: 15/744,593

(22) PCT Filed: Jul. 13, 2016
(86) PCT No.: PCT/EP2016/066590

§ 371 Date: Jan. 12, 2018

(30) Foreign Application Priority Data
Jul. 17, 2015 (FR) 1556791

(51) Int. Cl. C12N 1/12; A61K 36/02; A23K 10/16; A23K 20/147; A23L 17/60; A23L 33/135; A23L 33/195; A23K 50/75

(52) U.S. Cl. CPC C12N 1/12; A61K 36/02; A23K 10/16; A23K 20/147; A23V 2002/00; A23L 33/135; A23L 33/195; A23K 50/75; A23L 17/60

(57) ABSTRACT

The invention is in the field of cultures of microalgae, in particular thraustochytrids. The invention relates to a protein-rich biomass of thraustochytrids, to a method of obtaining said biomass, and to the uses thereof.

### FIELD OF THE INVENTION

The invention is in the field of cultures of microalgae, in particular thraustochytrids. It has as an object a thraustochytrid biomass which is rich in proteins, the process for obtaining said biomass and uses thereof in food.

### BACKGROUND ART

Several sources of plant proteins are known for use in food for human or animal consumption, directly or as dietary supplements, to provide animals and humans the amino acids necessary for their metabolism. These protein sources are intended as sources of amino acids available for the animals or humans once the food is ingested.

The best-known source of plant proteins used in animal feed is soybean, generally in the form of meal, which is the solid residue remaining after oil extraction. However, the use of soybean meal has several disadvantages associated with its origin. The meal is generally imported from countries which practice intensive soybean cultivation to the detriment of other plants that provide biodiversity. Furthermore, many countries promote the cultivation of genetically modified (GM) soybean varieties, which are found mixed with non-GM soybean in the meal, which does not help meet an increasing demand for genetically modified organism (GMO)-free food products of plant origin.

Animal feeds based on biomass composed of genetically modified photosynthetic microorganisms are known from WO 2010/051489. The effect of the recombinant enzyme produced by the GM microorganism is to degrade said biomass to make it compatible with animal feeding.

Other sources of plant proteins are known, notably spirulina and chlorella, used as dietary supplements for humans.

Spirulina, like chlorella, however, has the disadvantage of a low productivity, which precludes high-yield fermenter cultivation. If their cultivation makes it possible to meet a local and limited demand in conventional dietary supplements, it does not help meet the objective of wider, economically-viable industrial production of a source of dietary proteins the qualities of which will enable it to replace the common sources, such as soybean, in food for animal and human consumption.

Conversely, protists, known for their industrial production capacity in fermenters, have long been used to produce fats high in polyunsaturated fatty acids such as DHA or EPA, for example WO 97/37032, WO 2015/004402 or WO 2012/175027. However, the biomasses obtained, including after fat extraction, do not contain sufficient protein contents to permit their use as protein source in food, at the very least without costly additional protein-enrichment steps. Furthermore, the methods used for oil extraction may sometimes contaminate the remaining biomass, notably with organic solvents that make it unsuitable for consumption.

One of the goals of the invention is to provide a novel source of proteins for animal or human consumption which meets the objective of wide, economically-viable industrial production, the qualities of which will enable it to replace the common sources such as soybean.

The invention shows that, under certain culture conditions, thraustochytrids, known for their use in the production of oils with high polyunsaturated fatty acid contents (notably DHA, EPA) are microorganisms capable of producing a large amount of proteins, which can make them a source of dietary proteins similar to soybean, in particular for animal feed.

### DISCLOSURE OF THE INVENTION

Thus, the invention has as a first object a thraustochytrid biomass which may comprise, by weight relative to the weight of dry matter, at least 35% proteins, preferentially at least 45% proteins, which may range up to more than 60% proteins, indeed more than 75% proteins, in particular from 45% to 75% proteins.

The weight percentages of proteins may be expressed in terms of the proteins themselves or in terms of the amino acids contained in said proteins.

According to a variant of the invention, said biomass may further comprise, by weight relative to the weight of dry matter, less than 20% fat, preferentially less than 10% fat, more preferentially less than 7% fat.

Preferentially according to the invention, said biomass is a thraustochytrid biomass which may comprise, by weight relative to the weight of dry matter, at least 35% proteins, preferentially at least 45% proteins, very preferentially from 45% to 60% proteins and, still by weight relative to the weight of dry matter, less than 20% fat, preferentially less than 10% fat, more preferentially less than 7% fat.

The invention also relates to a process for producing a biomass as defined above and below, characterized in that it comprises:

a. a first step of culturing thraustochytrids in a suitable culture medium and under conditions which can promote the production of proteins at a level of at least 35% proteins by weight relative to the weight of dry matter and which, optionally, limits the production of fat to a level of less than 20% fat by weight relative to the weight of dry matter, until a culture density of at least 40 g/L dry matter, preferentially at least 60 g/L, more preferentially at least 80 g/L, is obtained;

b. a second step of recovering the biomass obtained in the first step by separating said biomass from the culture medium (harvesting); and, if need be

c. a third step of drying the biomass recovered in the second step.

The invention also relates to the use of a biomass as defined above or below, in the fields of human or animal cosmetics and food, and notably a food comprising such a biomass.

The invention also relates to the biomass according to the invention for use in therapy.

It also relates to cosmetic or pharmaceutical compositions for humans or animals and to food or food compositions for humans or animals, which comprise a biomass according to the invention.

### DETAILED DESCRIPTION OF THE INVENTION

According to the invention, the term “biomass” advantageously refers to a set of thraustochytrid cells produced by culturing the aforesaid protists, and having the levels of proteins and, optionally, fatty acids described in the present text, cells which may or may not retain their physical integrity.

It is thus understood that said biomass may comprise a quantity of degraded thraustochytrid cells ranging from 0% to 100%. The term “degraded” means that said thraustochytrid cells may have had their structure and/or composition modified. For example, they may have undergone a drying step or an oil harvesting step, the important thing being that the biomass comprising these cells has the levels of proteins and, optionally, of fatty acids described in the present text.

According to a preferred embodiment of the invention, the biomass has not undergone treatments that modify its amino acid composition during or after harvesting. That is, the treatments to which said biomass is subjected after harvesting do not alter the amino acid composition thereof. In particular, the biomass was not subjected to a step of enrichment in proteins and/or amino acids. That is, the proteins, peptides and amino acids contained in the biomass according to the invention derive only from the culture of thraustochytrids. It should be noted that proteins or amino acids not produced by thraustochytrids are likely to be present in the culture medium, notably in the case of preculture on medium comprising a yeast extract. The residual amounts of these proteins that may be present in the biomass, if any, will be in undetectable trace amounts, included in the definition of a biomass that does not undergo enrichment in proteins and/or amino acids.

According to a more preferred embodiment of the invention, the biomass has not undergone treatments that modify the amino acid and fat composition thereof. That is, the treatments to which said biomass is subjected after harvesting do not alter the amino acid and fat composition thereof. In particular, the relative composition of amino acids in relation to fat remains substantially constant.

It was observed in certain cases that non-degraded thraustochytrids in the biomass according to the invention have better properties of preservation and digestibility than degraded thraustochytrids. One of the preferred forms of the invention is a biomass comprising a substantially predominant amount of non-degraded thraustochytrids.

According to the invention, the term “degraded” refers to thraustochytrids the structural and/or chemical integrity of which may have been altered, such as for example lysed thraustochytrids, resulting for example from a homogenization process.

Nevertheless, it is evident than once produced, said biomass may be used raw, optionally dried, or subjected to any treatment necessary for the use thereof, notably homogenization.

According to the invention, said biomass may have, by weight relative to the weight of dry matter, a moisture content of 1% to 95%.

Preferentially, according to a first variant of the invention, said biomass may have, by weight relative to the weight of dry matter, a moisture content of 70% to 90%, preferentially 80% to 85%.

More preferentially, according to a second variant of the invention, said biomass may have, by weight relative to the weight of dry matter, a moisture content of 1% to 10%, preferentially 2% to 7%.

According to the invention, said thraustochytrids may be of the order Thraustochytriales, preferentially of the subclass Thraustochytriaceae, more preferentially of a genus which may be selected from the group comprising the genera Aurantiochytrium, Aplanochytrium, Botryochytrium, Japonochytrium, Oblongichytrium, Parietichytrium, Schizochytrium, Sicyoidochytrium, Thraustochytrium and Ulkenia.

The thraustochytrids are very preferentially non-genetically modified microorganisms. If GM thraustochytrids are used, they do not contain genes encoding one or more enzymes that make it possible to degrade or digest the biomass for use as food.

Advantageously, said thraustochytrids may be selected from the species Aplanochytrium kerguelense; Aplanochytrium minuta; Aplanochytrium stocchinoi; Aplanochytrium sp. PR24-1; Aurantiochytrium limacinum; Aurantiochytrium limacinum AB022107; Aurantiochytrium limacinum HM042909; Aurantiochytrium limacinum JN986842; Aurantiochytrium limacinum SL1101 JN986842; Aurantiochytrium mangrovei; Aurantiochytrium mangrovei DQ323157; Aurantiochytrium mangrovei DQ356659; Aurantiochytrium mangrovei DQ367049; Aurantiochytrium mangrovei CCAP 4062/2; Aurantiochytrium mangrovei CCAP 4062/3; Aurantiochytrium mangrovei CCAP 4062/4; Aurantiochytrium mangrovei CCAP 4062/5; Aurantiochytrium mangrovei CCAP 4062/6; Aurantiochytrium mangrovei CCAP 4062/1; Aurantiochytrium sp. AB052555; Aurantiochytrium sp. AB073308; Aurantiochytrium sp. ATCC PRA276 DQ836628; Aurantiochytrium sp. BL10 FJ821477; Aurantiochytrium sp. LY 2012 PKU Mn5 JX847361; Aurantiochytrium sp. LY2012 JX847370; Aurantiochytrium sp. N1-27; Aurantiochytrium sp. SD116; Aurantiochytrium sp. SEK209 AB290574; Aurantiochytrium sp. SEK217 AB290572; Aurantiochytrium sp. SEK 218 AB290573; Aurantiochytrium sp. 18W-13a; Botryochytrium radiatum; Botryochytrium radiatum Raghukumar 16; Botryochytrium radiatum SEK353; Botryochytrium sp.; Botryochytrium sp. BUTRBC 143; Botryochytrium sp. Raghukumar 29; Oblongichytrium minutum; Oblongichytrium multirudimentalis; Oblongichytrium sp.; Oblongichytrium sp. SEK347; Parieticytrium sarkarianum; Parieticytrium sarkarianum SEK351; Parieticytrium sarkarianum SEK364; Parieticytrium sp.; Parieticytrium sp. F3-1; Parieticytrium sp. H₁-14; Parieticytrium sp. NBRC102984; Phytophthora infestans; Schizochytrium aggregatum DQ323159; Schizochytrium aggregatum DQ356661; Schizochytrium aggregatum; Schizochytrium limacinum; Schizochytrium limacinum OUC166 HM042907; Schizochytrium mangrovei; Schizochytrium mangrovei FB1; Schizochytrium mangrovei FB3; Schizochytrium mangrovei FBS; Schizochytrium minutum; Schizochytrium sp. ATCC20888 DQ367050; Schizochytrium sp. KGS2 KC297137; Schizochytrium sp. SKA10 JQ248009; Schizochytrium sp. ATCC 20111; Schizochytrium sp. ATCC 20888; Schizochytrium sp. ATCC 20111 DQ323158*; Schizochytrium sp. ATCC 20888 DQ356660; Schizochytrium sp. ATCC 20889; Schizochytrium sp. ATCC 26185; Schizochytrium sp. BR2.1.2; Schizochytrium sp. BUCAAA 032; Schizochytrium sp. BUCAAA 093; Schizochytrium sp. BUCACD 152; Schizochytrium sp. BUCARA 021; Schizochytrium sp. BUCHAO 113; Schizochytrium sp. BURABQ 133; Schizochytrium sp. BURARM 801; Schizochytrium sp. BURARM 802; Schizochytrium sp. CCAP 4087/3; Schizochytrium sp. CCAP 4087/1; Schizochytrium sp. CCAP 4087/4; Schizochytrium sp. CCAP 4087/5; Schizochytrium sp. FJU-512; Schizochytrium sp. KH_{105;} Schizochytrium sp. KK17-3; Schizochytrium sp. KR-5; Schizochytrium sp. PJ10.4; Schizochytrium sp. SEK 210; Schizochytrium sp. SEK 345; Schizochytrium sp. SEK 346; Schizochytrium sp. SR21; Schizochytrium sp. T1001; Sicyoidochytrium minutum SEK354; Sicyoidochytrium minutum NBRC 102975; Sicyoidochytrium minutum NBRC 102979; Thraustochytriidae sp. BURABG162 DQ100295; Thraustochytriidae sp. CG9; Thraustochytriidae sp. LY2012 JX847378; Thraustochytriidae sp. MBIC11093 AB183664; Thraustochytriidae sp. NIOS1 AY705769; Thraustochytriidae sp. #32 DQ323161; Thraustochytriidae sp. #32 DQ356663; Thraustochytriidae sp. RT49 DQ323167; Thraustochytriidae sp. RT49 DQ356669; Thraustochytriidae sp. RT49; Thraustochytriidae sp. Thel2 DQ323162; Thraustochytriidae sp. Thel2; Thraustochytrium aggregatum; Thraustochytrium aggregatum DQ356662; Thraustochytrium aureum; Thraustochytrium aureum DQ356666; Thraustochytrium gaertnerium; Thraustochytrium kinnei; Thraustochytrium kinnei DQ323165; Thraustochytrium motivum; Thraustochytrium multirudimentale; Thraustochytrium pachydermum; Thraustochytrium roseum; Thraustochytrium sp. 13A4.1; Thraustochytrium sp. ATCC 26185; Thraustochytrium sp. BL13; Thraustochytrium sp. BL14; Thraustochytrium sp. BL2; Thraustochytrium sp. BL3; Thraustochytrium sp. BL4; Thraustochytrium sp. BLS; Thraustochytrium sp. BL6; Thraustochytrium sp. BL7; Thraustochytrium sp. BL8; Thraustochytrium sp. BL9; Thraustochytrium sp. BP3.2.2; Thraustochytrium sp. BP3.3.3; Thraustochytrium sp. caudivorum; Thraustochytrium sp. CHN-1; Thraustochytrium sp. FJN-10; Thraustochytrium sp. HK1; Thraustochytrium sp. HK10; Thraustochytrium sp. HK5; Thraustochytrium sp. HK8; Thraustochytrium sp. HK8a; Thraustochytrium sp. KK17-3; Thraustochytrium sp. KL1; Thraustochytrium sp. KL2; Thraustochytrium sp. KL2a; Thraustochytrium sp. ONC-T18; Thraustochytrium sp. PJA10.2; Thraustochytrium sp. TR1.4; Thraustochytrium sp. TRR2; Thraustochytrium striatum; Thraustochytrium striatum ATCC24473; Thraustochytrium striatum DQ323163; Thraustochytrium striatum DQ356665; Thraustochytrium visurgense; Ulkenia amoeboidea SEK 214; Ulkenia profunda; Ulkenia profunda BUTRBG 111; Ulkenia sp.; Ulkenia sp. ATCC 28207; Ulkenia visurgensis; Ulkenia visurgensis BURAAA 141; Ulkenia visurgensis ATCC 28208.

Preferentially according to the invention, the thraustochytrids may be selected from the genera Aurantiochytrium and Schyzochitrium, preferentially from the species

Aurantiochytrium mangrovei CCAP 4062/2 deposited 20 May 2014 at CCAP,

Aurantiochytrium mangrovei CCAP 4062/3 deposited 20 May 2014 at CCAP,

Aurantiochytrium mangrovei CCAP 4062/4 deposited 20 May 2014 at CCAP,

Aurantiochytrium mangrovei CCAP 4062/5 deposited 20 May 2014 at CCAP,

Aurantiochytrium mangrovei CCAP 4062/6 deposited 20 May 2014 at CCAP,

Aurantiochytrium CCAP 4062/1 deposited 21 June 2013 at CCAP,

Schizochytrium sp. CCAP 4087/3 deposited 20 May 2014 at CCAP,

Schizochytrium sp. CCAP 4087/1 deposited 28 February 2012 at CCAP,

Schizochytrium sp. CCAP 408⁷/₄ deposited 20 May 2014 at CCAP and

Schizochytrium sp. CCAP 4087/5 deposited 20 May 2014 at CCAP.

All deposits were made at CCAP (CULTURE COLLECTION OF ALGAE AND PROTOZOA (CCAP), SAMS Research Services Ltd., Scottish Marine Institute, OBAN, Argyl PA37 1QA United Kingdom) under the provisions of the Budapest Treaty.

According to a preferred variant of the invention, said biomass may be a biomass:
of thraustochytrids which may be selected from the genera Aurantiochytrium and Schyzochitrium, preferentially from the species Aurantiochytrium mangrovei CCAP 4062/2; Aurantiochytrium mangrovei CCAP 4062/3; Aurantiochytrium mangrovei CCAP 4062/4; Aurantiochytrium mangrovei CCAP 4062/5; Aurantiochytrium mangrovei CCAP 4062/6; Aurantiochytrium mangrovei CCAP 4062/1; Schizochytrium sp. CCAP 4087/3; Schizochytrium sp. CCAP 4087/1; Schizochytrium sp. CCAP 4087/4; Schizochytrium sp. CCAP 4087/5; and having:
a protein content, by weight relative to the weight of dry matter, of at least 35% proteins, preferentially at least 45% proteins, which may range up to more than 60% proteins, indeed more than 75% proteins, in particular from 45% to 75% proteins;
a fat content, by weight relative to the weight of dry matter, of less than 20%, preferentially less than 10%, very preferentially less than 7%; and
a moisture content before drying of 70% to 90%, preferentially 80% to 85%, or a moisture content after drying of 1% to 10%, preferentially 2% to 7%.

The invention also has as an object a process for producing a biomass as previously described which comprises:

a) a first step of culturing thraustochytrids in a suitable culture medium and under conditions which can promote the production of proteins at a level of at least 35% proteins, preferentially at least 45% proteins, which may range up to more than 60% proteins, indeed more than 75% proteins, in particular from 45% to 75% proteins, and which, optionally, limit the production of fat to a level of less than 20% fat by weight relative to the weight of dry matter, preferentially less than 10% fat, more preferentially less than 7% fat, until a culture density of at least 40 g/L dry matter, preferentially at least 60 g/L, more preferentially at least 80 g/L, is obtained;

b) a second step of recovering the biomass obtained in the first step by separating said biomass from the culture medium; and, if need be,

c) a third step of drying the biomass recovered in the second step.

Preferentially, step a) of culturing the thraustochytrids is carried out in a culture medium and under conditions suitable for promoting the production of proteins and for limiting the production of fat.

According to the invention, said suitable culture medium is preferably a chemically defined culture medium which comprises a carbon source, a nitrogen source, a phosphorus source and salts.

According to the invention, the term “chemically defined culture medium” refers to culture medium wherein the content of each element is known. Precisely, the invention is directed to a medium that may not comprise rich or complex organic matter. The expression “rich or complex organic matter” refers to unpurified organic matter, appearing as mixtures for which the exact composition and the concentrations of the various components of the mixture are not known with precision, not controlled, and may have a significant variability from one batch to another. By way of example of rich or complex organic matter, mention may be made of yeast extracts or peptones which are products of a protein hydrolysis reaction or also rich mineral matter such as for example marine mineral salts or other complex growth agents, not having a fixed concentration of each of their components.

According to the invention, said defined medium may comprise salts selected from calcium, cobalt, manganese, magnesium, zinc, nickel, copper, potassium, iron and sodium salts, and mixtures thereof.

Advantageously, said salts may be selected from calcium chloride, cobalt chloride, manganese chloride, magnesium sulfate, zinc sulfate, nickel sulfate, copper sulfate, potassium sulfate, iron sulfate, sodium molybdate, sodium selenite, sodium chloride and mixtures thereof.

According to a variant of the invention and according to the strains used, the medium may also comprise sodium chloride (NaCl), notably for certain strains of marine origin.

According to this variant, mention may be made by way of example of marine strains which may allow a culture medium which may comprise sodium chloride, strains of Schizochytrium sp., in particular Schizochytrium sp. CCAP 4062/3.

According to another variant of the invention and according to the strains used, the medium may not comprise sodium chloride (NaCl), at the very least may comprise a very small amount of sodium chloride, having less than 3.5 g/L, preferably less than 1 g/L, more preferentially less than 10 mg/L of sodium ions and less than 1 g/L, preferably less than 500 mg/L, more preferentially 200 mg/L of chloride ions.

According to this variant, mention may be made by way of example of strains which may allow a culture medium which may not comprise sodium chloride (NaCl), at the very least may comprise a very small amount of sodium chloride, strains of Aurantiochytrium mangrovei, in particular the strain Aurantiochytrium mangrovei CCAP 4062/5.

According to the invention, the carbon source of said defined medium may be one or more carbohydrates, one or more acetates, one or more alcohols, one or more complex molecules, or any mixture, in any proportion, of at least two of these sources.

According to the invention, said nitrogen source of said defined medium may be selected from one or more nitrate salts, one or more glutamate salts, one or more ammonium salts, urea, ammonia, or any mixture, in any proportion, of at least two of these sources.

According to the invention, the phosphorus source of said defined medium may be selected from phosphoric acid, phosphate salts, advantageously sodium hydrogen phosphate (Na₂HPO₄), or sodium dihydrogen phosphate (NaH₂PO₄), or potassium dihydrogen phosphate (KH₂PO₄), or potassium hydrogen phosphate (K₂HPO₄), or any mixture, in any proportion, of at least two of these sources.

According to a variant of the invention, said culture medium may comprise magnesium chloride, advantageously in tetrahydrate form (MgCl₂.4H₂O); calcium chloride, advantageously in dihydrate form (CaCl₂.2H₂O); cobalt chloride hexahydrate (CoCl₂.6H₂O); manganese(II) chloride tetrahydrate (MnCl₂.4H₂O); magnesium sulfate heptahydrate (MgSO₄.7H₂O); zinc sulfate heptahydrate (ZnSO₄.7H₂O); nickel sulfate hexahydrate (NiSO₄.6H₂O); copper sulfate pentahydrate (CuSO₄.5H₂O); potassium sulfate (K₂SO₄); iron sulfate heptahydrate (FeSO₄.7H₂O); boric acid (H₃BO₃); ethylenediaminetetraacetic acid in disodium dihydrate form (Na₂EDTA-2H₂O); sodium dihydrate molybdate (Na₂MoO₄.2H₂O); sodium selenite (Na₂SeO₃); as vitamin, thiamin, cobalamin or vitamin B12, pantothenate or vitamin B5; a carbon source; a nitrogen source; a phosphorus source.

According to a preferred form of the invention, in said culture medium, magnesium chloride may be at a concentration of 0.008 to 0.012 g/L, advantageously 0.009 to 0.011 g/L; calcium chloride may be at a concentration of 0.40 to 0.70 g/L, advantageously 0.50 to 0.60 g/L; cobalt chloride hexahydrate may be at a concentration of 0.00008 to 0.00013 g/L, advantageously 0.00009 to 0.00012 g/L; manganese(II) chloride tetrahydrate may be at a concentration of 0.008 to 0.013 g/L, advantageously 0.009 to 0.012 g/L; magnesium sulfate heptahydrate may be at a concentration of 6 to 10 g/L, advantageously 7 to 9 g/L; zinc sulfate heptahydrate may be at a concentration of 0.008 to 0.013 g/L, advantageously 0.009 to 0.012 g/L; nickel sulfate hexahydrate may be at a concentration of 0.004 to 0.007 g/L, advantageously 0.005 to 0.006 g/L; copper sulfate pentahydrate may be at a concentration of 0.005 to 0.009 g/L, advantageously 0.006 to 0.008 g/L; potassium sulfate may be at a concentration of 0.5 to 3.5 g/L, advantageously 1 to 3 g/L; iron sulfate heptahydrate may be at a concentration of 0.03 to 0.05 g/L, advantageously 0.035 to 0.045 g/L; boric acid may be at a concentration of 0.0155 to 0.0195 g/L, advantageously 0.0165 to 0.0185 g/L; ethylenediaminetetraacetic acid in disodium dihydrate form may be at a concentration of 0.10 to 0.14 g/L, advantageously 0.11 to 0.13 g/L; sodium dihydrate molybdate may be at a concentration of 0.00001 to 0.0003 g/L, advantageously 0.00005 to 0.0002 g/L; sodium selenite may be at a concentration of 0.00000015 to 0.000019 g/L, advantageously 0.00000016 to 0.00000018 g/L; thiamin may be at a concentration of 0.015 to 0.05 g/L, advantageously 0.025 to 0.04 g/L; cobalamin or vitamin B12 may be at a concentration of 0.0004 to 0.00065 g/L, advantageously 0.00045 to 0.00060 g/L; pantothenate or vitamin B5 may be at a concentration of 0.008 to 0.013 g/L, advantageously 0.009 to 0.012 g/L; the carbon source may be at a concentration of 45 to 65 g/L, advantageously 50 to 60 g/L; the nitrogen source may be at a concentration of 7 to 11 g/L, advantageously 8 to 10 g/L; the phosphorus source may be at a concentration of 2 to 6 g/L, advantageously 3 to 5 g/L.

Very preferentially according to the invention, in said culture medium, magnesium chloride is at a concentration of 0.0108 g/L; calcium chloride is at a concentration of 0.55 g/L; cobalt chloride hexahydrate (CoCl₂.6H₂O) is at a concentration of 0.000108 g/L; manganese(II) chloride tetrahydrate is at a concentration of 0.0108 g/L; magnesium sulfate heptahydrate is at a concentration of 8.01 g/L; zinc sulfate heptahydrate is at a concentration of 0.0108 g/L; nickel sulfate hexahydrate is at a concentration of 0.0056 g/L; copper sulfate pentahydrate is at a concentration of 0.0072 g/L; potassium sulfate is at a concentration of 2.09 g/L; iron sulfate heptahydrate is at a concentration of 0.04 g/L; boric acid is at a concentration comprised between 0.0155 and 0.0195 g/L of 0.0175 g/L; ethylenediaminetetraacetic acid in disodium dihydrate form is at a concentration of 0.12 g/L; sodium dihydrate molybdate is at a concentration of 0.000108 g/L; sodium selenite is at a concentration of 0.000000173 g/L; thiamin is at a concentration of 0.032 g/L; cobalamin or vitamin B12 is at a concentration of 0.00052 g/L; pantothenate or vitamin B5 is at a concentration of 0.0108 g/L; the carbon source is at a concentration of 55 g/L; the nitrogen source is at a concentration of 9 g/L; the phosphorus source is at a concentration of 4 g/L.

According to the invention, the first culture step a) of the process may be carried out in co-called “batch” discontinuous mode, in so-called “fed batch” semi-continuous mode or in continuous mode.

According to a particular embodiment of the invention, the first step is divided into two sub-steps, a first growth sub-step a1) in the suitable culture medium followed by a second production sub-step a2) wherein one or more carbon source, nitrogen source and/or phosphorus source enrichment solutions may be added to the culture medium, simultaneously or successively, so as to maintain in the culture medium nitrogen and phosphorus levels that do not limit growth.

According to a preferred embodiment, the growth sub-step a1) is carried out until a concentration of carbon source, more particularly of glucose, of less than 20 g/L is obtained.

According to another embodiment, the growth sub-step al) is carried out until a culture density of at least 20 g/L, preferentially at least 40 g/L, more preferentially at least 60 g/L, even more preferentially at least 80 g/L, is obtained.

The non-limiting level of nitrogen source in step a2) is advantageously 0.5 to 5 g/L, preferentially 0.5 to 2 g/L, and the non-limiting level of phosphorus source is advantageously 0.5 to 5 g/L, preferentially 0.5 to 2 g/L. The carbon source content sought for this step a2) may be 0 to 200 g/L, notably 5 or 10 to 50 g/L. Preferentially, the carbon source content in sub-step a2) is 0 to 50 g/L, more preferentially 0 to 10 g/L.

Preferentially, the culture of which the first step a) is divided into two sub-steps a1) and a2) as defined above is carried out in so-called “fed batch” semi-continuous mode.

The suitable methods and culture media for enabling the growth of thraustochytrids at densities greater than 20 g/L, and in particular greater than 80 g/L, are well-known to persons skilled in the art.

According to the invention, the culture may be carried out by any known culture technique, for example in flasks or in a reactor, but also in fermenters or in any container suitable for growing protists, particularly thraustochytrids, such as for example “raceway”-type basins, provided that said technique makes it possible to carry out the required culture conditions.

Preferentially, the culture is carried out in fermenters according to the known methods for culturing protists in fermenters.

According to the invention, the second step b) of the process for recovering said biomass may be carried out under suitable conditions for obtaining a biomass which may have the moisture content sought.

Said recovery of the protists may be carried out by any technique allowing recovery of the biomass, notably methods of filtration, gravimetric or under reduced pressure, centrifugation, decantation, or even methods of precipitation followed by gravimetric filtration.

The invention also has as an object a biomass which can be obtained by the process according to the invention as described above in all its variants.

The invention also has as an object the use of a biomass as described above in the fields of human or animal cosmetics, pharmaceuticals or food.

It particularly relates to the use of a thraustochytrid biomass according to the invention, as described above and below, for improving animal performance. This improvement in performance may be evaluated, in particular, by measuring consumption, weight gain or feed conversion ratio.

In animal feeding, the feeding of livestock, in particular intensive livestock operations, will be distinguished from that of domestic animals or pets or so-called “leisure” animals, such as aquarium fish or aviary or caged birds.

The term “livestock” refers notably to grazing animals (particularly cattle raised for meat, milk, cheese and leather; sheep raised for meat, wool and cheese; caprines), pigs, rabbits, poultry (chickens, hens, turkeys, ducks, geese, etc.), members of the horse family (ponies, horses, foals), animals intended to support human activities (transport, leisure) or the feeding thereof, aquatic animals (for example fish, shrimp, oysters and mussels). However, the feeding of fish up to the alevin stage may be distinguished from that of raised fish, including the feed and feed compositions intended therefor.

Domestic animals, pets and leisure animals, which also include mammals, ruminants or not, will be distinguished from birds and fish. They include in particular dogs and cats.

The invention also has as an object a food, or food composition, for humans or animals, which may comprise a biomass according to the invention as described above. The term “food” refers to any composition which may be used as food for humans or animals, in particular livestock.

According to the invention, the food may comprise only the biomass, optionally dried, optionally transformed, or the biomass, optionally dried, optionally transformed, mixed with any other additive, carrier or support, used in the field of food for human or animal consumption, such as for example food preservatives, dyes, flavor enhancers, pH regulators, or also pharmaceutical additives such as for example growth hormones, antibiotics.

The present invention concerns in particular feeds for animals and more particularly for livestock. These feeds typically appear in the form of flours, pellets or slop into which the biomass according to the invention is incorporated. The term “feed” refers to anything that may be used to feed animals.

For intensive animal breeding operations, the feeds may comprise, in addition to the algal biomass, a nutritional base and nutritional additives. The essential part of the animal's feed ration thus consists of the “nutritional base” and the algal biomass. This base consists, by way of example, of a mixture of cereals, proteins and fats of animal and/or plant origin.

Nutritional bases for animals are adapted to the feeding of these animals and are well- known to persons skilled in the art. In the context of the present invention, these nutritional bases comprise for example corn, wheat, pea and soybean. These nutritional bases are adapted to the needs of the various animal species for which they are intended. These nutritional bases may already contain nutritional additives such as vitamins, mineral salts and amino acids.

The additives used in animal feed may be added to improve certain characteristics of the feeds, for example to enhance the flavor thereof, to make the raw materials of the feeds more digestible for the animals or to protect the animals. They are frequently used in large-scale intensive breeding operations.

The additives used in animal feeds can be divided into the following subcategories in particular (source: EFSA):
technological additives: for example, preservatives, antioxidants, emulsifiers, stabilizers, acidity regulators and silage additives;
sensory additives: for example, flavors, dyes,
nutritional additives: for example, vitamins, amino acids and trace elements;
zootechnical additives: for example, digestibility enhancers, intestinal flora stabilizers;
coccidiostats and histomonostats (pesticides).

In an embodiment, the invention relates to livestock feeds comprising 1% to 60%, preferably 1% to 20%, quite preferentially 3% to 8% of a dried biomass obtained by the process according to the invention.

In another embodiment, the invention relates to livestock feeds comprising 1% to 40%, preferably 5% to 10% of a non-dried biomass obtained by the process of the invention.

According to a particular embodiment of the invention, the feed is intended for livestock, in particular cattle, sheep, pigs, rabbits, poultry and horses.

According to another particular embodiment of the invention, the feed is intended for aquatic animals, in particular fish, at least up to the alevin stage, indeed including farmed fish.

According to another particular embodiment of the invention, the feed is intended for domestic animals, pets and/or leisure animals.

Finally, according to another embodiment of the invention, the food composition is intended for humans.

The invention also has as an object a cosmetic or pharmaceutical composition for humans or animals comprising a biomass according to the invention as described above.

According to the invention, the cosmetic or pharmaceutical composition may comprise only the biomass, optionally dried, optionally transformed, or the biomass, optionally dried, optionally transformed, mixed with any other additive, carrier or support used in the field of cosmetics or pharmaceuticals, such as, for example, preservatives, dyes, pH regulators.

The invention also has as an object the use of the biomass as described above in therapy, as well as in the prevention and treatment of malnutrition.

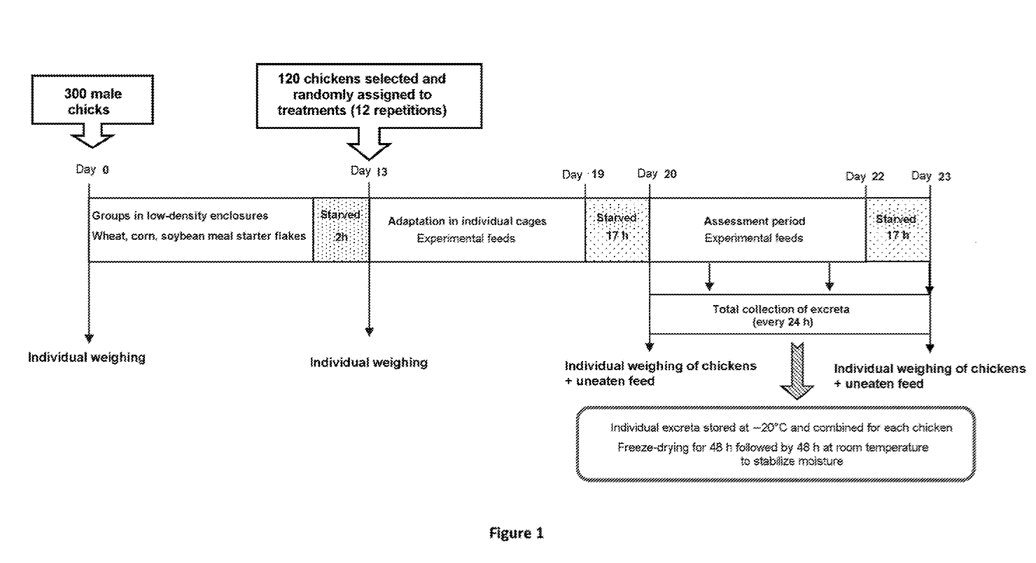

FIG. 1: Experimental design of the measurement of apparent metabolizable energy (AME) in 23-day-old chickens; (test 14ALG069)

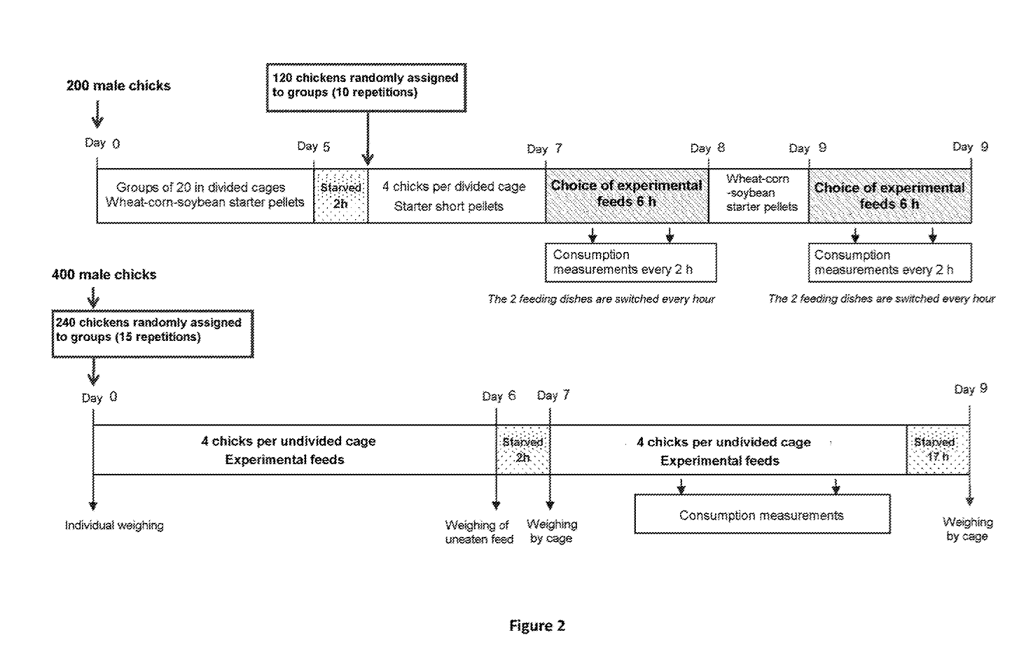

FIG. 2: Experimental design of the choice and consumption test in chicks (7 and 9 days old); (test 14ALG081)

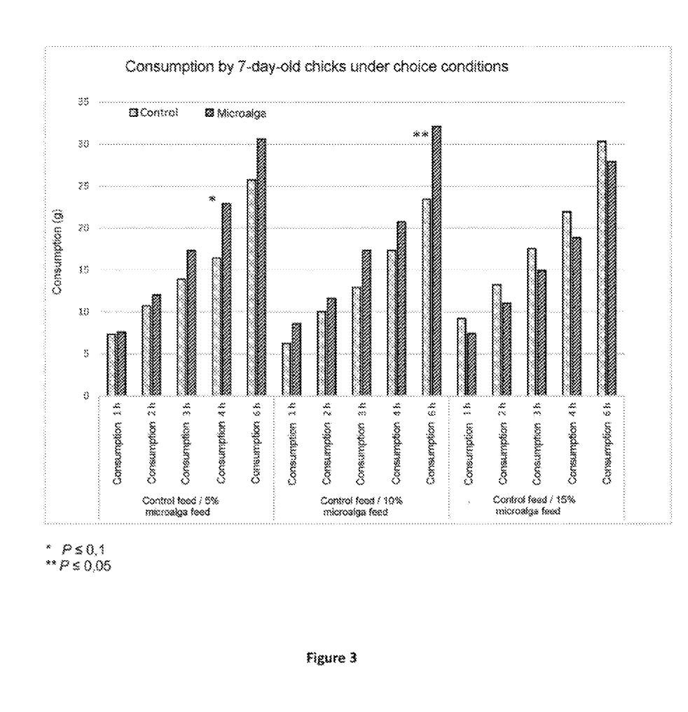

FIG. 3: Measurement of feed consumption at 7 days of age under choice conditions (corn-soybean feed vs feed substituted with 5%, 10% and 15% of the microalga tested) (test 14ALG081)

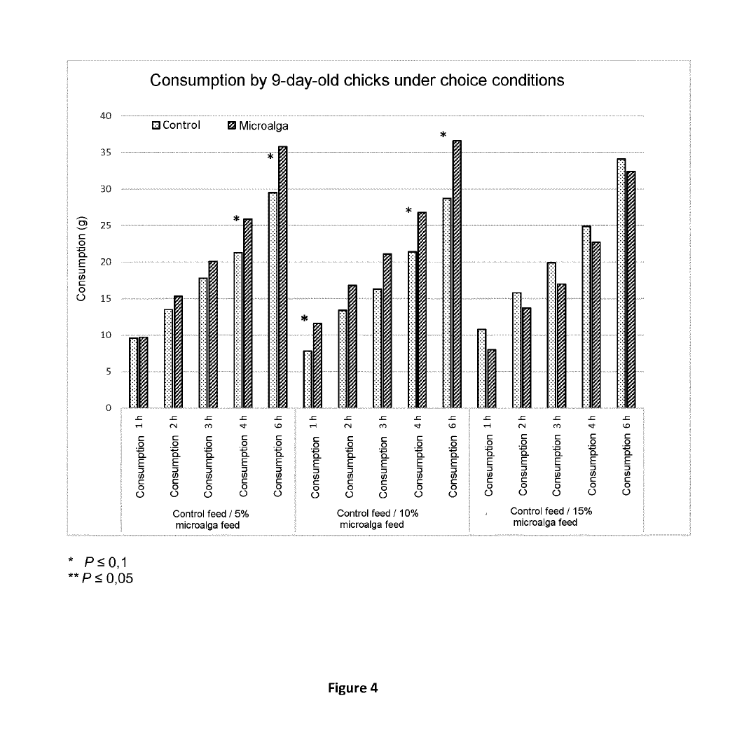

FIG. 4: Measurement of feed consumption at 9 days of age under choice conditions (corn-soybean feed vs feed substituted with 5%, 10% and 15% of the microalga tested) (test 14ALG081)

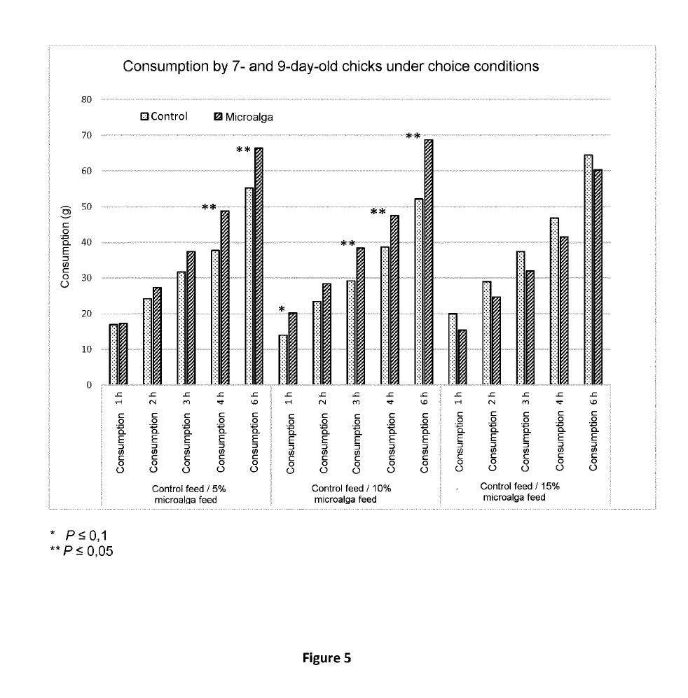

FIG. 5: Measurement of mean relative feed consumption over the 2 tests (at 7 d and 9 d) under choice conditions (corn-soybean feed vs feed substituted with 5%, 10% and 15% of the microalga tested) (test 14ALG081)

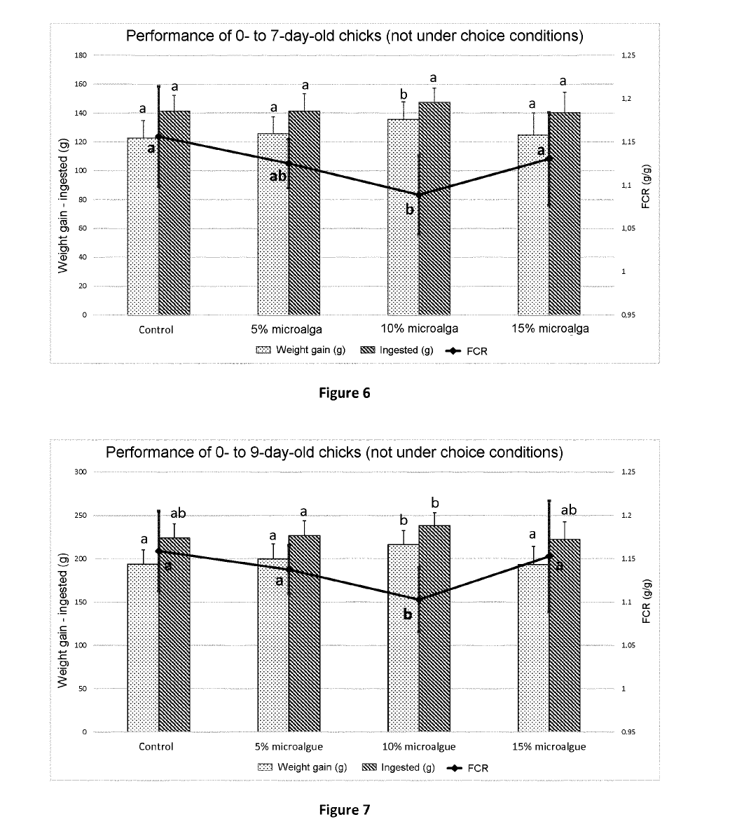

FIG. 6: Measurement of growth performance (consumption, weight gain, feed conversion ratio) of 0- to 7-day-old chicks fed a control corn-soybean diet vs feed substituted with 5%, 10% and 15% of the microalga tested (not under choice conditions) (test 14ALG081)

FIG. 7: Measurement of growth performance (consumption, weight gain, feed conversion ratio) of 0- to 9-day-old chicks fed a control corn-soybean diet vs feed substituted with 5%, 10% and 15% of the microalga tested (not under choice conditions) (test 14ALG081)

### EXAMPLES

Other aspects and features of the invention will become apparent upon reading the following examples and in the appended figures describing same.

FIG. 1 shows the experimental design of the measurement of apparent metabolizable energy (AME) in 23-day-old chickens; (test 14ALG069). Approximately 300 1-day-old male chicks (Ross PM3) were placed in the same low-density enclosure and fed a standard starter containing wheat, corn and soybean meal. At 13 days of age the chickens are starved for 2 hours before being weighed and distributed by weight group. One hundred twenty chickens are thus selected, placed in individual metabolic cages and assigned to one of the experimental treatments according to their weight (20 repetitions per diet).

Feed and water are distributed ad libitum throughout the test. The experimental feeds are provided as 3.2-mm-diameter pellets. After a 7-day period of adaptation to the diets and the metabolic cages, a total excreta collection period, flanked by 17-hour fasts, was carried out for 3 days, from day 20 to day 23. The excreta are collected individually each day, combined and stored at −20° C. At the end of the test the collected excreta are freeze-dried then left at room temperature for a water-uptake phase (48 h) in order to stabilize the moisture content before weighing, grinding (0.5 mm) and analyses.

FIG. 2 shows the experimental design of the choice and consumption tests in chicks (7 and 9 days old); (test 14ALG081). Two experimental tests were carried out in parallel.

1) Test 1—choice test: For the test with choice of feed, approximately 200 1-day-old male chicks (Ross PM3) were placed in groups of about 20 in divided metabolic cages and were fed a standard starter containing wheat, corn and soybean meal. At 6 days of age the chickens are starved for 2 hours before being weighed and distributed by weight group. One hundred twenty chickens are thus selected, placed by groups of 4 into 30 divided metabolic cages and assigned at day 7 to one of the experimental treatments according to their weight (10 repetitions per treatment). Each cage contains two feeding dishes containing different feeds, corresponding to the following three experimental treatments:
Treatment 1: corn-soybean starter (feeding dish 1) and 5% microalga (feeding dish 2)
Treatment 2: corn-soybean starter (feeding dish 1) and 10% microalga (feeding dish 2)
Treatment 3: corn-soybean starter (feeding dish 1) and 15% microalga (feeding dish 2)

Feed and water are dislributed ad libitum throughout the test. The experimental feeds are provided as 3.2-mm-diameter pellets. Consumption is measured at T₀₊₁ₕ, T₀₊₂ₕ, T₀₊₃ₕ, T₀₊₄ₕ and T₀₊₆ₕ at 7 and 9 days of age, with the feeding dishes in the cages being switched every hour. Between the two consumption measurements (day 8), the animals receive the wheat-corn-soybean starter.

2) Test 2—consumption measurement: in a second test, the chicks have access to only one type of feed, optionally supplemented with microalga (FIG. 6). Approximately 400 1-day-old male chicks (Ross PM3) were weighed and distributed by weight group. Two hundred forty selected animals are placed by groups of 4 at day 1 in 60 undivided cages in blocks of homogeneous weight (15 blocks of 4 cages) with one feeding dish per cage, each with an experimental feed (15 repetitions per feed). In each cage, the 4 chicks are identified individually. Feed and water are distributed ad libitum throughout the test. The experimental feeds are corn-soybean starter supplemented with 0%, 5%, 10% or 15% microalga, also used in test 1 with choice of feed. Individual live weights are measured at 1, 7 and 9 days of age.

Uneaten feeds are weighed and consumption per cage is measured at 7 and 9 days of age. From 0 to 7 days of age, the animals fed the feed containing 10% microalga have a significantly improved weight gain compared to the control and to the other two supplemented diets.

FIG. 3 shows the measurement of feed consumption at 7 days of age under choice conditions (corn-soybean feed vs feed substituted with 5%, 10% and 15% of the microalga tested) (test 14ALG081);

FIG. 4 shows the measurement of feed consumption at 9 days of age under choice conditions (corn-soybean feed vs feed substituted with 5%, 10% and 15% of the microalga tested) (test 14ALG081);

FIG. 5 shows the measurement of mean relative food consumption over the 2 tests (at 7 d and 9 d) under choice conditions (corn-soybean feed vs feed substituted with 5%, 10% and 15% of the microalga tested) (test 14ALG081);

FIG. 6 shows the measurement of growth performance (consumption, weight gain, feed conversion ratio) of 0- to 7-day-old chicks fed a control corn-soybean diet vs feed substituted with 5%, 10% and 15% of the microalga tested (not under choice conditions) (test 14ALG081);

FIG. 7 shows the measurement of growth performance (consumption, weight gain, feed conversion ratio) of 0- to 9-day-old chicks fed a control corn-soybean diet vs feed substituted with 5%, 10% and 15% of the microalga tested (not under choice conditions) (test 14ALG081).

### Example 1

### Strain Cultures

Strain Precultures:

An Aurantiochytrium mangrovei preculture is prepared on a shaker table (140 rpm) in a temperature-controlled enclosure (26° C.), in preculture medium, containing 4 g of yeast extract as nitrogen source, and 30 g of glucose as carbon source. After 48 hours of incubation the cells are centrifuged for 5 minutes at 3000 g and the cell pellet is rinsed with preculture medium containing neither yeast extract nor any other source of mineral or organic nitrogen. The purpose of this operation is to avoid any supply of Na⁺ in the main culture via the addition of yeast extract. The preculture corresponds to 1/100 (v/v) of the culture volume of the main solution. In the case of strain Schizochytrium sp. CCAP 4062/3,27 g/L of NaCl is added to this medium.

Culture Monitoring:

Total biomass concentration is monitored by measuring the dry mass (filtration on GF/F filter, Whatman, then oven drying, at 105° C., for at least 24 h before weighing).

Analyses of total lipid and FAME contents are carried out according to the methods classically described in the literature (Folch et al., A simple method for the isolation and purification of total lipids from animal tissues. Folch J. et al., J Biol Chem. 1957 May; 226(1):497-509.; Yokoyama et al., Taxonomic rearrangement of the genus Schizochytrium sensu lato based on morphology, chemotaxonomic characteristics, and 18S rRNA gene phylogeny (Thraustochytriaceae, Labyrinthulomycetes): emendation for Schizochytrium and erection of Aurantiochytrium and Oblongichytrium gen. November; Mycoscience, 2007 Vol. 48, pp. 199-211).

### Example 1.1

### Comparative

Aurantiochytrium mangrovei CCAP 4062/5 cultures are prepared in 1- to 2-L fermenters (bioreactors) for use with computer-controlled automated systems. The composition of the culture medium and those of the addition solutions are as follows:

| Main solution | Concentration (g/L) |
|---|---|
| KCl | 0.36 |
| H₃BO₃ | 0.175 |
| MgSO_{4•}7H₂O | 6.750 |
| CaCl₂•2H₂O | 0.55 |
| KNO₃ | 0.04667 |
| KH₂PO₄•7H₂O | 0.30940 |
| Na₂EDTA•2H₂O | 3.094 · 10⁻³ |
| ZnSO₄•7H₂O | 7.3 · 10⁻⁵ |
| CoCl₂•6H₂O | 1.6 · 10⁻⁵ |
| MnCl₂•4H₂O | 5.4 · 10⁻⁴ |
| Na₂MoO_{4•}2H₂O | 1.48 · 10⁻⁶ |
| Na₂SeO₃ | 1.73 · 10⁻⁶ |
| NiSO₄•6H₂O | 2.98 · 10⁻⁶ |
| CuSO₄•5H₂O | 9.8 · 10⁻⁶ |
| EDTA-Fe | 0.03 |
| Carbon | g/L |
| Glucose | 55 |
| Nitrogen | g/L |
| (NH4)₂SO₄ | 7 |

Addition after autoclave

| Vitamins | g/L |
|---|---|
| Thiamin | 8.0 · 10⁻³ |
| Vitamin B12 | 1.3 · 10⁻⁴ |
| Pantothenate | 2.7 · 10⁻³ |

| Addition solution 1 | Concentration (g/L) |
|---|---|
| K₂SO₄ | 31.9 |
| MgSO₄•7H₂O | 25.8 |
| KH₂PO₄•7H₂O | 61.38 |
| FeSO₄•7H₂O | 0.61 |
| (NH₄)₂SO₄* | 138.24 |
| MnCl₂•4H₂O | 0.165 |
| ZnSO₄•7H₂O | 0.165 |
| CoCl₂•6H₂O | 1.6 · 10⁻³ |
| Na₂MoO₄•2H₂O | 1.6 · 10⁻³ |
| CuSO₄•5H₂O | 0.11 |
| NiSO₄•6H₂O | 8.6 · 10⁻² |
| Na₂EDTA•2H₂O | 1.81 |
| Thiamin | 0.49 |
| Vitamin B12 | 8.0 · 10⁻² |
| Pantothenate | 0.1656 |
| Addition solution 2 | Concentration g/L |
| Glucose | 750 |
| KH₂PO₄ | 6.4 |
| (NH₄)2SO₄* | 34 |

The system is adjusted to pH 6 by adding base (NaOH, or KOH) and/or acid (sulfuric acid solution). The culture temperature was set to 26° C. Dissolved oxygen pressure was regulated in the medium throughout the culture, by shaking speed (250-1200 rpm), air flow rate (0.25-1 vvm), or oxygen flow rate (0.1-0.5 vvm). The regulatory parameters, integrated into the controller, made it possible to maintain a constant pO₂ of 5% to 30%. Culture time was 20 to 100 hours, preferably 25 to 96 hours, for example 50 hours.

During the culture, three additions of addition solution 1 were carried out, as well as additions of solution 2 in order to maintain glucose concentrations between 200 mM and 500 mM.

### Example 1.2

### Invention

Aurantiochytrium mangrovei CCAP 4062/5 cultures are prepared in fermenters as essentially described in Example 1.1.

The procedure is modified in terms of the mode of pH regulation by addition of ammonia (NH₄OH) to avoid the large supply of Na+or K+related to pH regulation by sodium hydroxide or potassium hydroxide, and which may pose problems in terms of the development of animal feed. Part of the nitrogen necessary for culturing the cells is supplied via the regulation of pH by ammonia (NH₄OH).

The medium used for this example is described in Table 1 below. Unlike the medium described in Example 1.1, this chemically defined medium makes it possible to sustain growth throughout the culture without nutritional limitations.

TABLE 1

| | Concentration (g/L) |
|---|---|
| Magnesium chloride | 1.08 · 10⁻² |
| Calcium chloride | 0.55 |
| Cobalt chloride hexahydrate (CoCl₂•6H₂O) | 1.08 · 10⁻⁴ |
| Manganese(II) chloride tetrahydrate | 1.08 · 10⁻² |
| Magnesium sulfate heptahydrate | 8.01 |
| Zinc sulfate heptahydrate | 1.08 · 10⁻² |
| Nickel sulfate hexahydrate | 5.60 · 10⁻³ |
| Copper sulfate pentahydrate | 7.20 · 10⁻³ |
| Potassium sulfate | 2.09 |
| Iron sulfate heptahydrate | 4.00 · 10⁻² |
| Boric acid | 1.75 · 10⁻² |
| Ethylenediaminetetraacetic acid disodium dihydrate | 0.12 |
| Sodium dihydrate molybdate | 1.08 · 10⁻⁴ |
| Sodium selenite | 1.73 · 10⁻⁷ |
| Thiamin | 3.20 · 10⁻² |
| Cobalamin or vitamin B12 | 5.20 · 10⁻⁴ |
| Pantothenate or vitamin B5 | 1.08 · 10⁻² |
| Glucose (carbon source) | 55.00 |
| Ammonium sulfate (nitrogen source) | 9.00 |
| Potassium dihydrogen phosphate (phosphorus | 4.00 |
| source) | |
| Defoamer | 0.40 |

The physicochemical parameters are controlled during the culture by means of integrated regulators, with pH maintained around a setting of 5, temperature set to 30° C., and pO₂ maintained around a setting of 30% until maximum shaking and air flow rate values are reached.

During the culture, additions of addition medium, as described in Table 2 below, are carried out so as to maintain the glucose concentration in the culture medium between 20 g/L and 50 g/L.

TABLE 2

| | | |
|---|---|---|
| Glucose (carbon source) | 688 | g/L |
| Ammonium sulfate (nitrogen source) | 76 | g/L |
| Potassium dihydrogen phosphate (phosphorus source) | 39 | g/L |

### Example 1.3

### Invention

Aurantiochytrium mangrovei CCAP 4062/5 cultures are prepared in fermenters under culture conditions as essentially described in Example 1.1.

The procedure is modified in terms of the media and the mode of addition thereof.

The cultures are started in batch mode on the medium presented in Table 3 below.

TABLE 3

| | | Initial concentrtion | |
|---|---|---|---|
| | Compound | in the medium | |
| Glucose + | Glucose•1H₂O | 55.000 | g/L |
| Sea salt | Sea salt | 15.000 | g/L |
| Mineral | MgSO₄•7H₂O | 1.683 | g/L |
| salts | K₂SO₄ | 2.080 | g/L |
| | KH₂PO₄ | 4.000 | g/L |
| | MnCl₂•4H₂O | 10.800 | mg/L |
| | ZnSO₄•7H₂O | 1.080 | mg/L |
| | CoCl₂•6H₂O | 0.011 | mg/L |
| | Na₂MoO₄•2H₂O | 0.108 | mg/L |
| | CuSO₄•5H₂O | 0.720 | mg/L |
| | NiSO₄•6H₂O | 0.560 | mg/L |
| | FeSO₄•7H₂O | 40.000 | mg/L |
| Vitamins | Thiamin | 32.000 | mg/L |
| | Vitamin B₁₂ | 0.520 | mg/L |
| | Pantothenate | 10.800 | mg/L |
| Nitrogen | (NH₄)₂SO₄ | 9.000 | g/L |
| EDTA | Na₂EDTA | 0.096 | g/L |

The continuous culture mode is then started once the biomass reaches 20 g/L in the medium. The feed solution supplied continuously is that described in Table 4 below.

TABLE 4

| Compound | Feed solution (g/L) |
|---|---|
| Glucose•1H₂O | 110.000 |
| Sea salt | 15.000 |
| MgSO₄•7H₂O | 4.959 |
| K₂SO₄ | 6.129 |
| KH₂PO₄ | 11.786 |
| MnCl₂•4H₂O | 3.182 · 10⁻² |
| ZnSO₄•7H₂O | 3.182 · 10⁻² |
| CoCl₂•6H₂O | 3.2 · 10⁻⁴ |
| Na₂MoO₄•2H₂O | 3.200 · 10⁻⁴ |
| CuSO₄•5H₂O | 2.121 · 10⁻² |
| NiSO_{4•}6H₂O | 1.650 · 10⁻² |
| FeSO₄•7H₂O | 1.179 · 10⁻¹ |
| Thiamin | 9.429 · 10⁻² |
| Vitamin B12 | 1.530 · 10⁻³ |
| Pantothenate | 3.182 · 10⁻² |
| (NH₄)₂SO₄ | 26.518 |
| EDTA | 4.092 · 10⁻¹ |

Preliminary tests showed that the dilution rate used directly effects the protein content of the biomass (results not shown). For this example, the dilution rate used is 0.13 h⁻¹, which corresponds to half the maximum growth rate of the strain under the culture conditions used.

### Example 1.4

### Comparative

Schizochytrium sp. CCAP 4062/3 cultures are prepared in fermenters as essentially described in Example 1.1, with culture medium supplemented with 27 g/L and 35.6 g/L NaCl for the initial fermenter medium and the addition medium, respectively.

### Example 1.5

### Invention

Schizochytrium sp. CCAP 4062/3 cultures are prepared in fermenters as essentially described in Example 1.2, with culture medium supplemented with 27 g/L and 35.6 g/L NaCl for the initial fermenter medium and the addition medium, respectively.

Results

| | DM (g/L) | AA/DM (%) | Fat/DM (%) |
|---|---|---|---|
| Soybean (beans)* | — | 40.43 | 21.80 |
| Soybean (meal)* | — | 48.80 | 0.55 |
| Example 1.1 (comparative) | 90.0 | 30.00 | 34.30 |
| Example 1.2 (invention) | 67.0 | 57.90 | 6.10 |
| Example 1.3 (invention) | 60.1 | 35.23 | 6.56 |
| Example 1.4 (comparative) | 87.0 | 20.44 | 45.97 |
| Example 1.5 (invention) | 79.9 | 49.66 | 13.6 |

DM: Dry matter;
AA: Amino acids;
*Values in % relative to 100% DM according to the values given by the USDA

It is noted that the culture processes of the art intended for the production of fats enriched in polyunsaturated fatty acids do not make it possible to obtain compositions with a high protein content (expressed in wt % of amino acids relative to dry matter).

### Example 2

### Animal Nutrition

### Example 2.1

### Materials and Methods

Strain Aurantiochytrium Mangrovei CC4062/5 was cultivated under the conditions of Example 1.2. These conditions are those used in the following examples unless otherwise mentioned.

2.1.1—Biomass Composition:

The microalgal biomass was first analyzed for its proximate composition ((moisture (EAU-H internal method adapted to Regulation EC 152/2009 of 27 Jan. 2009 (103° C./4h)—SN), fats by hydrolysis (Regulation EC 152/2009 of 27 Jan. 2009—Procedure B—SN), crude proteins estimated on the basis of the sum of the total amino acid concentrations (Directive 98/64/EC, 9/3/99—Standard NF EN ISO 13903), mineral substances (Regulation EC 152/2009 of 27 Jan. 2009—SN)), and gross energy (NF EN 14918), total fiber (AOAC 985.29—SN), insoluble walls (according to the method of Carre and Brillouet (1989, Determination of water-insoluble cell walls in feeds: interlaboratory study. Journal Association of Official Analytical Chemists, 72, 463-467), calcium (NF EN ISO 6869—March 2002—CT), phosphorus (Regulation EC 152/2009 of 27 Jan. 2009—CT), chloride (internal method adapted from ISO 1841-2—July 1996—SN), potassium (internal method—ICP 05/07—SN), sodium (internal method—ICP 05/07—SN) and total amino acid concentrations.

2.1.2—Measurement of Amino Acid Digestibility in Caecectomized Cockerels:

In parallel, this microalga is evaluated in vivo for the measurement of amino acid digestibility in caecectomized cockerels (Green et al., 1987). The animals (adult Isa Brown cockerels) were force-fed a mash composed of one of the ingredients tested (microalga tested or a control soybean meal) and supplemented with wheat starch to obtain a protein level of 18% corresponding to the animals' need. Twelve cockerels housed in individual cages were used per treatment. Each animal received on average 171.2±14.5 g of mash after being starved for 24 h. The excreta collected up to 48 h after force-feeding were collected by group of 4 cockerels such that 3 excreta per treatment are freeze-dried. They were then left at room temperature for a water-uptake phase (48 h) in order to stabilize the moisture content, then weighed, ground (0.5 mm) and analyzed by the Terpstra method (Terpstra and de Hart, 1974) for their non-uric acid nitrogen content and for their total amino acid concentrations (JEOL AminoTac JLC-500N). Endogenous losses of amino acids were previously determined by evaluating a protein-free diet based on the same experimental design. The corrected results of the basal endogenous losses are thus expressed as a standardized ileal digestibility coefficient of amino acids, calculated and subjected to statistical analysis as follows:

$${DIS ( X )}_{F} = [ \frac{{X}_{F} * {I}_{D} * {L}_{F} - {X}_{E} * {Q}_{E} + \frac{{X}_{END} * {I}_{D}}{10}}{{X}_{F} * {I}_{D} * {L}_{F}} ] * 1000$$

with DIS (X)_{F}: standardized ileal digestibility coefficient (%) of amino acid X of raw material F; X_{F}: concentration (g/100 g) of the amino acid in the raw material; X_{E}: concentration (g/100 g) of the amino acid in the excreta; I_{D}: amount of feed consumed (g); Q_{E}: amount excreted (g); L_{F}: incorporation ratio of the raw material in the feed; X_{END}: endogenous losses of amino acid X (g/100 g).

The experimental results are first analyzed for ingredient effect according to an ANOVA procedure (XLSTAT 2010.4.02© Addinsoft 1995-2010), according to the following model with a 95% confidence interval:

Yᵢμ+aᵢ

with:
Y=parameter
μ=mean
aᵢ=ingredient effect

For each significant difference, the means are analyzed by a pairwise Fisher's LSD test.

### Example 2.2

### Composition of Strain CC4062/5 Cultivated for 49 h

Strain CC4062/5 was cultivated as mentioned above. The culture was stopped at 49 h, producing several kilograms of microalgal biomass for the purpose of carrying out a preliminary digestibility test in the animals. This biomass was dried on a drying cylinder and has the form of flakes. It is identified as “strain 4, 49-h culture”.

The biochemical composition results for the “microalga, strain 4, 49-h culture” are presented in Table 5 below and are expressed as both gross and corrected dry matter (DM) content.

TABLE 5
(test 14ALG058): Composition of the microalga tested
(strain 4, 49-h culture) vs a control soybean meal

| | Concentrations | | Concentrations | |
|---|---|---|---|---|
| | (gross %) | | (% of dry matter) | |
| | | Soybean | | Soybean |
| | μA | meal | μA | meal |
| Dry matter, % | 94.8 | 89.5 | — | — |
| Moisture, % | 5.2 | 10.5 | — | — |
| Sum of amino acids | 50.8 | 46.9 | 53.4 | 52.4 |
| Fats | 10.6 | 5.0 | 11.2 | 5.6 |
| Crude fiber | n/d | 4.2 | n/d | 4.7 |
| Mineral substances | 7.3 | 5.8 | 7.7 | 6.5 |
| Starch | <1.0 | n/a | <1.1 | n/a |
| Total sugars | 1.3 | n/d | 1.4 | n/d |
| ADF | n/d | 3.7 | n/d | 4.1 |
| NDF | n/d | 7.2 | n/d | 8.0 |
| Total dietary fiber (TDF) | 16.5 | n/d | 17.4 | n/d |
| Water-insoluble cell | 4.9 | n/d | 5.2 | n/d |
| wall (WICW) | | | | |
| Calcium | 0.118 | 0.371 | 0.124 | 0.414 |
| Phosphorus | 1.448 | 0.598 | 1.527 | 0.668 |
| Chloride (Cl⁻) | 0.150 | n/d | 0.158 | n/d |
| Potassium | 1.983 | n/d | 2.092 | n/d |
| Sodium | 0.068 | n/d | 0.072 | n/d |
| | (kcal/kg) | | (kcal/kg DM) | |
| Gross energy | 4870 | 4486 | 5137 | 5012 |

n/d: not determined
n/a: not applicable
μA: microalga

These results show that the microalga has a total nitrogenous matter (sum of amino acids) and mineral substances composition slightly higher than that of the control soybean meal. The mineral content of the biomass tested is comparable to the lowest concentrations published, ranging from 7% to 43% DM depending on the family, genus and species of the microalga. The phosphorus and calcium contents are inversely proportional with 2.3 times more phosphorus and 3.3 times less calcium in the microalga relative to the control soybean meal. The “strain 4, 49-h culture” tested does not contain starch reserves and its total sugar concentration remains low (1.4% DM) compared to the raw materials conventionally used in animal nutrition. In contrast, its fat content is two times higher than that of soybean meal (11.2% vs 5.6% DM) and its gross energy 125 kcal/kg DM higher.

Microalgae walls have different compositions and structures than those of raw materials of plant origin, and render unsuitable the fiber analyses conventionally used. Also, analysis of total dietary fiber (TDF) (Prosky et al., 1988) has the advantage of being less restrictive and the “microalga, strain 4, 49-h culture” has a total fiber percentage of 17.4% DM (which, by way of comparison, is about what is analyzed in rye or barley grain) with a water-insoluble cell wall (WICW) residue measured at 5.2% DM.

The total protein concentrations reported in Tables 4 to 8 derive from the sum of amino acids themselves assayed according to the method described above. The sum of amino acids—as estimator of the total proteins of the “microalga, strain 4, 49-h culture”—corresponds to 53.4% DM, with more precisely 21.5% DM of essential amino acids and 32.0% DM of nonessential amino acids. Table 6 shows the amino acid concentrations analyzed in the microalga and the soybean meal and expressed as gross values or relative to the sum of amino acids.

The results reflect that the microalga has a relatively balanced amino acid profile, which agrees with the work comparing the amino acid profiles of different microalgae with those of so-called conventional protein sources (Becker, 2007).

Generally, the essential amino acid concentrations of the microalga are slightly higher than those measured in the soybean meal (21.5% DM and 24.0% DM, respectively).

TABLE 6
(test 14ALG058): Total nitrogen and amino acid concentrations (gross %),
amino acid concentrations relative to the sum of amino acids (% sum AA),
standardized ileal digestibility coefficients of amino acids (%) measured
in caecectomized cockerels, and digestible amino acid concentrations (gross
%) of the microalga tested vs a control soybean meal.

| | | | | | Standardized ileal | | | Digestible | |
|---|---|---|---|---|---|---|---|---|---|
| | [AA] | | AA/sum AA | | digestibility of AA | | | [AA] | |
| | (gross %) | | (%) | | (%) | | | (gross %) | |
| | μA | SM | μA | SM | μA | SM | p-value | μA | SM |
| Dry matter | 94.8 | 89.5 | — | — | — | — | — | — | — |
| Total nitrogen | 10.6 | 7.8 | — | — | 80.1 ± 0.3 | 89.6 ± 0.7 | <0.0001 | 8.49 | 6.97 |
| Sum AA | 50.8 | 46.9 | — | — | 78.4 ± 1.0 | 88.5 ± 1.0 | 0.001 | 39.79 | 41.53 |
| Essential AA | 20.4 | 21.5 | | | | | | | |
| Methionine | 0.85 | 0.64 | 1.67 | 1.36 | 85.6 ± 1.3 | 90.9 ± 1.6 | 0.023 | 0.73 | 0.58 |
| Lysine | 2.30 | 2.87 | 4.53 | 6.12 | 84.9 ± 1.3 | 87.6 ± 0.8 | 0.068 | 1.95 | 2.51 |
| Threonine | 1.71 | 1.88 | 3.37 | 4.01 | 80.7 ± 1.8 | 86.0 ± 1.7 | 0.038 | 1.38 | 1.62 |
| Tryptophan | 0.55 | 0.67 | 1.08 | 1.43 | 82.9 ± 1.7 | 90.3 ± 1.3 | 0.009 | 0.46 | 0.61 |
| Arginine | 6.83 | 3.42 | 13.44 | 7.29 | 61.0 ± 0.8 | 93.2 ± 0.8 | <0.0001 | 4.17 | 3.19 |
| Isoleucine | 1.48 | 2.30 | 2.91 | 4.90 | 85.4 ± 1.2 | 88.7 ± 1.2 | 0.048 | 1.26 | 2.04 |
| Leucine | 2.52 | 3.69 | 4.96 | 7.87 | 86.7 ± 1.1 | 88.5 ± 1.2 | 0.182 | 2.18 | 3.27 |
| Valine | 2.01 | 2.35 | 3.96 | 5.01 | 87.5 ± 1.6 | 89.0 ± 1.5 | 0.389 | 1.76 | 2.09 |
| Histidine | 0.66 | 1.18 | 1.30 | 2.52 | 88.7 ± 0.6 | 89.9 ± 1.4 | 0.327 | 0.59 | 1.06 |
| Phenylalanine | 1.52 | 2.48 | 2.99 | 5.29 | 84.6 ± 1.5 | 89.8 ± 1.0 | 0.014 | 1.29 | 2.23 |
| Nonessential AA | 30.3 | 25.5 | | | | | | | |
| Cystine | 0.55 | 0.63 | 1.08 | 1.34 | 60.9 ± 3.7 | 79.7 ± 2.0 | 0.003 | 0.34 | 0.50 |
| Serine | 1.87 | 2.52 | 3.68 | 5.37 | 76.7 ± 1.6 | 88.5 ± 1.3 | 0.001 | 1.43 | 2.23 |
| Proline | 1.07 | 2.14 | 2.11 | 4.56 | 84.3 ± 2.1 | 87.9 ± 1.3 | 0.107 | 0.90 | 1.88 |
| Alanine | 2.17 | 2.07 | 4.27 | 4.41 | 84.9 ± 1.1 | 84.9 ± 1.1 | 0.992 | 1.84 | 1.76 |
| Aspartic ac. | 3.57 | 5.49 | 7.03 | 11.71 | n/d | 85.9 ± 0.6 | n/d | n/d | 4.71 |
| Glutamic ac. | 18.10 | 8.81 | 35.63 | 18.78 | 73.4 ± 0.7 | 90.4 ± 0.7 | 0.0001 | 13.29 | 7.96 |
| Glycine | 1.81 | 2.01 | 3.56 | 4.29 | 78.3 ± 2.3 | 84.3 ± 1.6 | 0.036 | 1.42 | 1.69 |
| Tyrosine | 1.18 | 1.78 | 2.32 | 3.80 | 84.2 ± 1.7 | 89.5 ± 0.6 | 0.015 | 0.99 | 1.59 |

μA: microalga;
SM: soybean meal;
[AA]: amino acid concentration

### Example 2.3

### Measurement of Biomass Amino Acid Digestibility (Strain CC4062/5 Cultivated for 49 h)

The results concerning standardized ileal digestibility (SID) of amino acids measured in caecectomized cockerels, as well as the digestible amino acid concentrations, are presented in Table 6. The digestibilities of nitrogen and the sum of amino acids of the microalga are 80.1±0.3% and 78.4±1.0%, respectively, which are 9.5 and 10.1 points lower than the control soybean meal. The arginine and cystine SIDs of the microalga have the lowest results, respectively 61.0±0.8% and 60.9 35 3.7%. Generally, the digestibility coefficients of the essential amino acids of the microalga range from 80.7±1.8% (threonine SID) to 88.7±0.6% (histidine SID). Lysine digestibility is measured at 85.6±1.3%. These coefficients are about 1.2 (histidine SID) to 7.4 (tryptophan SID) points lower than those measured for the control soybean meal. However, they reflect high digestibility coefficients, making it possible to consider the evaluated microalga as a protein source of good quality (i.e., they are significantly higher than the microalgae protein digestibility values reported in the literature by about 55% to 77% (Henman et al., 2012).

### Example 2.4

### Composition of Strain CC4062/5 Cultivated for 22 h

Strain CC4062/5 was cultivated under the same conditions but for a period of 22 h. It is identified as “strain 4, 22-h culture”.

As before, this biomass was dried on a drying cylinder and has the physical form of flakes.

It was analyzed for its composition of proximates, gross energy, total fiber, insoluble walls, calcium, phosphorus, chloride, potassium, sodium and total amino acid concentrations.

The biochemical composition of this “microalga, strain 4, 22-h culture” is presented in Table 7. The results are expressed as both gross and corrected dry matter (DM) content.

The “microalga, strain 4, 22-h culture” has a total nitrogenous matter composition (sum of amino acids) 2.3 and 1.3 points higher than those of the control soybean meal and the “microalga, strain 4, 49-h culture”.

Its mineral substance content increases relative to a longer culture but remains at a concentration level which is in the lower range of the values published in the literature (Skrede et al., 2011).

The phosphorus/calcium ratio is unbalanced. The phosphorus and calcium contents are inversely proportional with 3.6 times more phosphorus and 3.6 times less calcium in the microalga relative to the control soybean meal. The optimized strain 4 (22-h culture) contains 1.5 times more total phosphorus than during a 49-h culture. The fat content is higher than that of the soybean meal (8% vs 5.6% DM), and the results confirm that neither starch nor total sugars represent a form of energy storage in this strain 4. Gross energy is measured at 4641 kcal/kg DM, or 371 kcal/kg DM less vs the control soybean meal.

The total dietary fiber (TDF) of the “microalga, strain 4, 22-h culture” represents 12.7% of DM with a water-insoluble cell wall (WICW) residue measured at 2.1% DM. In parallel, the residue deduced as follows: R (%)=100—mineral substances (%)—total nitrogenous matter (%)—fat (%)—starch (%)—sugars (%) indicates that the TDF content is 15.1 points lower than that of R thus estimated at 27.8% DM. These observations agree with other work (Lieve et al., 2012) reporting about 20% to 30% of the amount of the dried biomass not explained by the sum of the contents of mineral substances, lipids, proteins and carbohydrates.

The “microalga, strain 4, 22-h culture” has 2 points higher fat than the soybean meal taken in comparison (Table 7).

TABLE 7
(test 14ALG065): Composition of the microalga tested
(strain 4, 22-h culture) vs a control soybean meal

| | AA concentrations | | AA concentrations | |
|---|---|---|---|---|
| | (gross %) | | (% dry matter) | |
| | | Soybean | | Soybean |
| | μA | meal | μA | meal |
| Dry matter | 94.6 | 89.5 | — | — |
| Moisture | 5.4 | 10.5 | — | — |
| Sum of amino acids | 51.7 | 46.9 | 54.7 | 52.4 |
| Fat | 7.6 | 5.0 | 8.0 | 5.6 |
| Crude fiber | n/d | 4.2 | n/d | 4.7 |
| Mineral substances | 9.0 | 5.8 | 9.5 | 6.5 |
| Starch | <0.5 | n/a | <0.5 | <0.6 |
| Total sugars | <0.5 | n/d | <0.5 | <0.6 |
| ADF | n/d | 3.7 | n/d | 4.1 |
| NDF | n/d | 7.2 | n/d | 8.0 |
| Total dietary fiber (TDF) | 12.0 | n/d | 12.7 | n/d |
| Water-insoluble cell | 2.0 | n/d | 2.1 | n/d |
| wall (WICW) | | | | |
| Calcium | 0.109 | 0.371 | 0.115 | 0.415 |
| Phosphorus | 2.279 | 0.598 | 2.410 | 0.668 |
| Chloride (Cl⁻) | 0.160 | n/d | 0.169 | n/d |
| Potassium | 2.238 | n/d | 2.366 | n/d |
| Sodium | 0.161 | n/d | 0.170 | n/d |
| | (kcal/kg) | | (kcal/kg DM) | |
| Gross energy | 4390 | 4486 | 4641 | 5012 |

n/d: not determined;
n/a: not applicable
μA: microalga

The sum of amino acids amounts to 54.7% of DM, with more precisely 23.9% DM of essential amino acids and 30.8% DM of nonessential amino acids. Table 8 presents the amino acid concentrations analyzed in the microalga and the soybean meal and expressed as gross values or relative to the sum of amino acids. The results reflect that the microalga has a relatively balanced amino acid profile compared to the control soybean meal. The glutamic acid, arginine and aspartic acid of the “microalga, strain 4, 22-h culture” are present in higher proportions with values (% sum AA) of 26.75%, 9.48% and 8.26%, respectively. The lysine content (% sum AA) is 5.94% in reference to that of the control soybean meal (6.12%). Arginine, methionine and, to a lesser extent, threonine contribute more strongly to the microalga protein tested than to that of the soybean meal (9.48% vs 7.29%, 2.13% vs 1.36%, and 4.27% vs 4.01%, respectively). The microalga cultivated for 22 h has 2.4 points more essential acids than a 49-h culture, or the same content as that of the control soybean meal. Except for arginine, essential amino acids are present in a larger proportion of the sum of amino acids.

The “microalga, strain 4, 22-h culture” has methionine, arginine and threonine contents higher than those of the soybean, whereas the lysine and valine contents are equivalent or slightly higher in the microalgal biomass.

### Example 2.5

### Measurement of Biomass Amino Acid Digestibility (Strain CC4062/5 Cultivated for 22 h)

The measurement of amino acid digestibility in caecectomized cockerels (Green et al., 1987) was performed identically to that described above (see Example 2.3).

The results concerning standardized ileal digestibility (SID) of amino acids of the “microalga, strain 4, 22-h culture” measured in caecectomized cockerels, as well as the digestible amino acid concentrations, are presented in Table 8. The digestibilities of nitrogen and the sum of amino acids of the microalga are 85.7 ±0.7% and 87.1 ±0.4%, respectively, or 3.2 (p=0.008) and 0.9 (p=0.190) points lower than the control soybean meal. Compared to the 49-h culture, the digestibility of the sum of amino acids of this production of “microalga, strain 4, 22-h culture” is significantly increased by 8.7 points. The concentrations of digestible lysine, methionine, arginine, threonine and valine are higher than those of the control soybean meal. They reflect a good quality of the protein of the microalga tested, superior or equivalent to that of soybean meal.

The results of measurement of amino acid digestibility in cockerels show that the “microalga, strain 4, 22-h culture” is a protein source of good quality. The digestibility of the proteins (sum of amino acids) is similar to that of soybean meal, which is the protein source most used worldwide for feeding monogastrics.

Furthermore, the drying process used does not appear to be a factor that denatures the quality of the protein, given the high digestibility coefficients measured for lysine.

It is important to note that the quality of the protein is significantly improved when the results of the “microalga, strain 4, 49-h culture” are compared with those of the “microalga, strain 4, 22-h culture”. Notably, protein digestibility increases by about 9 points.

TABLE 8
(test 14ALG065): Total nitrogen and amino acid concentrations (gross %),
amino acid concentrations relative to the sum of amino acids (% sum AA),
standardized ileal digestibility coefficients of amino acids (%) measured
in caecectomized cockerels, and digestible amino acid concentrations (gross
%) of the microalga tested vs a control soybean meal.

| | | | | | Standardized ileal | | | Digestible | |
|---|---|---|---|---|---|---|---|---|---|
| | [AA] | | AA | | digestibility of AA | | | [AA] | |
| | (gross %) | | (%) | | (%) | | | (gross %) | |
| | μA | SM | μA | SM | μA | SM | p-value | μA | SM |
| Dry matter | 94.6 | 89.5 | — | — | — | — | — | — | — |
| Total nitrogen | 11.5 | 7.8 | — | — | 85.7 ± 0.7 | 88.9 ± 0.5 | 0.008 | 9.86 | 6.92 |
| Sum AA | 51.7 | 46.9 | — | — | 87.1 ± 0.4 | 88.0 ± 0.7 | 0.190 | 45.06 | 41.2 |
| Essential AA | 22.6 | 21.5 | | | | | | | |
| Methionine | 1.10 | 0.64 | 2.13 | 1.36 | 90.0 ± 0.4 | 91.5 ± 0.3 | 0.015 | 0.99 | 0.59 |
| Lysine | 3.07 | 2.87 | 5.94 | 6.12 | 87.9 ± 0.2 | 85.6 ± 0.3 | 0.0001 | 2.70 | 2.46 |
| Threonine | 2.21 | 1.88 | 4.27 | 4.01 | 87.3 ± 0.3 | 85.2 ± 1.0 | 0.045 | 1.93 | 1.60 |
| Tryptophan | 0.68 | 0.67 | 1.32 | 1.43 | 86.0 ± 0.2 | 89.3 ± 1.1 | 0.013 | 0.59 | 0.60 |
| Arginine | 4.90 | 3.42 | 9.48 | 7.29 | 79.9 ± 1.1 | 93.0 ± 0.5 | <0.0001 | 3.92 | 3.18 |
| Isoleucine | 1.98 | 2.30 | 3.83 | 4.90 | 88.2 ± 0.3 | 88.5 ± 0.5 | 0.526 | 1.75 | 2.04 |
| Leucine | 3.31 | 3.69 | 6.40 | 7.87 | 89.0 ± 0.2 | 88.5 ± 0.7 | 0.341 | 2.95 | 3.27 |
| Valine | 2.57 | 2.35 | 4.97 | 5.01 | 87.1 ± 0.2 | 85.6 ± 1.2 | 0.153 | 2.24 | 2.01 |
| Histidine | 0.82 | 1.18 | 1.59 | 2.52 | 88.7 ± 2.1 | 89.6 ± 0.7 | 0.578 | 0.73 | 1.06 |
| Phenylalanine | 1.96 | 2.48 | 3.79 | 5.29 | 87.4 ± 0.4 | 89.8 ± 0.7 | 0.012 | 1.71 | 2.23 |
| Nonessential AA | 29.1 | 25.5 | | | | | | | |
| Cystine | 0.65 | 0.63 | 1.26 | 1.34 | 69.4 ± 1.2 | 79.1 ± 2.3 | 0.006 | 0.45 | 0.50 |
| Serine | 2.09 | 2.52 | 4.04 | 5.37 | 83.0 ± 0.5 | 87.7 ± 1.0 | 0.004 | 1.73 | 2.21 |
| Proline | 1.66 | 2.14 | 3.21 | 4.56 | 87.9 ± 0.8 | 87.2 ± 1.2 | 0.519 | 1.46 | 1.87 |
| Alanine | 2.80 | 2.07 | 5.42 | 4.41 | 88.8 ± 0.3 | 84.7 ± 1.0 | 0.005 | 2.49 | 1.75 |
| Aspartic ac. | 4.27 | 5.49 | 8.26 | 11.71 | n/d | 85.7 ± 0.8 | n/d | n/d | 4.71 |
| Glutamic ac. | 13.83 | 8.81 | 26.75 | 18.78 | 84.8 ± 0.7 | 90.5 ± 0.6 | 0.001 | 11.72 | 7.97 |
| Glycine | 2.34 | 2.01 | 4.53 | 4.29 | 81.8 ± 0.9 | 83.3 ± 0.5 | 0.125 | 1.92 | 1.67 |
| Tyrosine | 1.48 | 1.78 | 2.86 | 3.80 | 86.7 ± 0.1 | 89.4 ± 0.9 | 0.016 | 1.28 | 1.59 |

n/d: not determined;
μA: microalga;
SM: soybean meal;
[AA]: amino acid concentration

### Example 2.6

### Determination of Apparent Metabolizable Energy (AME) and Nitrogen-Corrected Apparent Metabolizable Energy (AMEn) of Strain CC4062/5 Cultivated for 22 h

The measurement of apparent metabolizable energy (AME) of the microalga “microalga, strain 4, 22-h culture” was performed in 3-week-old chickens (Bourdillon et al., 1990). The so-called substitution method was applied starting with a control diet composed of corn-soybean base and premix. Like the other raw materials, the microalga, ground on a 3-mm grid, was incorporated in increasing proportions of 4%, 8%, 12%, 16% and 20% in substitution for the corn-soybean mixture, while keeping the premix constant in the diets.

Table 9 presents the formula of the control corn-soybean feed as well as the specifications thereof (crude protein=18%, apparent metabolizable energy=3200 kcal/kg). The feeds substituted with the microalga are optimally balanced (notably in their protein to energy ratio) on the basis of assumptions regarding AME, digestible amino acids, and bioavailable phosphorus made for the microalga.

TABLE 9
(test 14ALG069): Formula of the
experimental feed supplement used

| | D1 |
|---|---|
| | Basal Corn-Soybean |
| Corn | 67.673 |
| Soybean meal 48% | 19.742 |
| Corn gluten 60 | 4.766 |
| Soybean oil | 3.139 |
| Premix | 4.680 |
| Microalga | 0 |
| Crude protein | 18 |
| Fat | 6.201 |
| Crude fiber | 3.029 |
| Mineral substances | 2.013 |
| Lysine | 0.80 |
| Methionine | 0.33 |
| Met + Cys | 0.664 |
| Threonine | 0.686 |
| Tryptophan | 0.173 |
| Arginine | 1.066 |
| Calcium | 0.1 |
| Total phosphorus | 0.333 |
| Available phosphorus | 0.078 |
| Sodium | 0.092 |
| AME, kcal/kg | 3200 |

Diet 1: basal corn-soybean (95.32%)+vitamin and mineral supplement (VMS) (4.68%)

Diet 2: basal corn-soybean (91.32%)+VMS (4.68%)+microalga (4%)

Diet 3: basal corn-soybean (87.32%)+VMS (4.68%)+microalga (8%)

Diet 4: basal corn-soybean (83.32%)+VMS (4.68%)+microalga (12%)

Diet 5: basal corn-soybean (79.32%)+VMS (4.68%)+microalga (16%)

Diet 6: basal corn-soybean (75.32%)+VMS (4.68%)+microalga (20%)

This incorporation gradient makes it possible to evaluate the AME value of the microalga by simple linear regression. The experimental design is described in FIG. 1. The dry matter values of the diets were determined after grinding and on feed pellets at the beginning and end of the assessment period. Finally, the complete feeds and the feces were analyzed individually for their gross energy (GE) content by calorimetry.

The AMEn of the feeds is calculated as follows:

$$EMA ( kcal \text{/} kg DM ) = \frac{( I \times GEi - E \times GEf )}{I}$$

$$EMAn ( kcal \text{/} kg DM ) = EMA - 8.22 \frac{( WG \times 0.18 ) / 6.25}{I}$$

with I: amount of feed ingested (kg DM); E: amount of excreta (kg); GEi and GEf: gross energy of the feed ingested (kcal/kg DM) and of the feces (kcal/kg); WG: weight gain during the assessment period. The AME values were first nitrogen-corrected on the basis of 18% protein contained in animal tissue and by using the factor 8.22 kcal/kg N (Hill and Anderson, 1958). In parallel, nitrogen-correction calculations were also made based on assay of total nitrogen in the excreta. The two methods giving identical results, it is the latter method which is considered in this report. The outliers (±2.5 standard deviations from the mean) were removed before treatment by analysis of variance (ANOVA, SAS 9.1.3© 2002-2003 by SAS Institute Inc., Cary, N.C., USA) according to a factorial design. Dry matter digestibility (dDM) and the apparent digestibility coefficients of nitrogen (DCa nitrogen), phosphorus (DCa phosphorus), calcium (DCa calcium) and fat (DCa fat) are calculated in the same way for each diet.

These results were analyzed by linear regression (XLSTAT 2010.4.02© Addinsoft 1995-2010), according to the following model:)

Yᵢ=β₀+βⱼXᵢⱼ+ϵᵢ

With: Yᵢ=value observed for the dependent variable for observation i,
β₀=intercept
βⱼ=slope of the regression line
Xᵢⱼ=value of variable j for observation i (% incorporation of the microalga),
ϵᵢ=model error.

The value of the parameter for 100% incorporation of the microalga was extrapolated from the prediction model, thus making it possible to estimate AME and AMEn and the apparent digestibility coefficient of phosphorus of the microalga.

Table 10 presents the dry matter digestibility and nitrogen-corrected apparent metabolizable energy (AMEn) results of the control corn-soybean feed as well as the substituted diets D2 to D6. The apparent digestibility coefficients of nitrogen (DCa nitrogen), phosphorus (DCa phosphorus), calcium (DCa calcium) and fat (DCa fat) were estimated by the same method.

TABLE 10
(test 14ALG069): Dry matter digestibility, energy digestibility, and apparent digestibility coefficient of the experimental feeds substituted
with 0%, 4%, 8%, 12%, 16% and 20% of the microalga tested (strain 4, 22-h culture), respectively, and measured in 23-day-old chickens

| | Experimental feeds | | | | | | |
|---|---|---|---|---|---|---|---|
| | Diets | | | | | | |
| | | D2 | D3 | D4 | D5 | D6 | |
| | D1 | BC-S + | BC-S + | BC-S + | BC-S + | BC-S + | |
| | BC-S | 4% μA | 8% μA | 12% μA | 16% μA | 20% μA | p-value |
| dDM (%)¹ | 75.10 ± 1.20a | 74.41 ± 1.09ab | 73.53 ± 1.64b | 71.64 ± 1.43c | 70.17 ± 1.22d | 69.56 ± 2.05d | <0.0001 |
| AMEn (kcal/kg DM)² | 3398.0 ± 53a | 3379.4 ± 42a | 3331.8 ± 64b | 3273.6 ± 61c | 3218.9 ± 53d | 3171.4 ± 72e | <0.0001 |
| GE/AMEn (%)³ | 78.35 ± 1.34a | 78.05 ± 1.25a | 77.71 ± 1.72a | 76.19 ± 1.56b | 75.00 ± 1.56C | 74.72 ± 1.84c | <0.0001 |
| DCa nitrogen (%)⁴ | 61.79 ± 3.75a | 60.24 ± 2.44ab | 58.80 ± 4.87b | 56.48 ± 3.15c | 53.97 ± 2.26d | 53.90 ± 3.44d | <0.0001 |
| DCa phosphorous (%)⁵ | 38.00 ± 4.67a | 40.78 ± 4.36b | 41.42 ± 4.87b | 36.96 ± 4.89a | 37.77 ± 3.38a | 37.28 ± 3.83a | 0.004 |
| DCa calcium (%)⁶ | 26.84 ± 5.41a | 34.32 ± 5.85b | 37.86 ± 5.91ab | 34.90 ± 4.61bc | 36.74 ± 4.55bcd | 38.94 ± 5.01d | <0.0001 |
| DCa fat (%)⁷ | 85.88 ± 4.2ab | 87.17 ± 1.88ac | 88.59 ± 2.53c | 85.93 ± 3.33a | 83.97 ± 3.37bd | 83.86 ± 3.14d | <0.0001 |

B C-S: basal corn-soybean;
μA: microalga
¹Dry matter digestibility (%);
²Nitrogen-corrected apparent metabolizable energy (AMEn);
³Gross energy relative to AMEn (%);
⁴Apparent digestibility coefficient of nitrogen;
⁵Apparent digestibility coefficient of phosphorus;
⁶Apparent digestibility coefficient of calcium,
⁷Apparent digestibility coefficient of fat (%)

The dry matter digestibility of the various diets is strongly correlated with their AMEn values. The AMEn of corn-soybean diet D1 is measured at 3398±53 kcal/kg DM. The results show that the AMEn value of diet D2 is equivalent to that of the corn-soybean control and reflect that the 4% substitution with the microalga does not impact the energy digestibility of the diet. In contrast, and starting from an 8% or higher substitution of microalga, AMEn and DCa nitrogen decrease linearly with increasing percentages of substitution (p<0.0001).

The addition of 4% to 16% microalga shows a reduction in the feed conversion ratio (FCR) over the period 13-23 days. It should also be noted that the chickens tend to consume less (except for D2) for a similar weight gain, however, regardless of the percentage of incorporation of the microalga and higher than that measured for the control corn-soybean diet (Table 11).

TABLE 11
(test 14ALG069): Weight gain, consumption, and feed conversion ratio of the animals over the period 13 to 23 days fed
diets substituted with 0%, 4%, 8%, 12%, 16% and 20% of the microalga tested (strain 4, 22-h culture), respectively.

| | Experimental feeds | | | | | |
|---|---|---|---|---|---|---|
| | | D2 | D3 | D4 | D5 | D6 |
| | D1 | BC-S + | BC-S + | BC-S + | BC-S + | BC-S + |
| | BC-S | 4% μA | 8% μA | 12% μA | 16% μA | 20% μA |
| Weight gain (g) | 460 ± 58.8 a | 496 ± 36.5 b | 495 ± 90.0 b | 516 ± 56.6 b | 510 ± 37.5 b | 493 ± 44.1 ab |
| Consumption (g) | 749 ± 82.9 a | 756 ± 50.3 a | 713 ± 118.8 ab | 722 ± 68.0 ab | 701 ± 51.9 be | 665 ± 52.8 c |
| Feed conversion ratio (g/g) | 1.63 ± 0.063 a | 1.53 ± 0.048 b | 1.44 ± 0.067 c | 1.41 ± 0.060 d | 1.37 ± 0.043 de | 1.35 ± 0.042 e |

B C-S: basal corn-soybean;
μA: microalga

The AME and AMEn values of the “microalga, strain 4, 22-h culture” (Table 12) are measured at 2785 kcal/kg DM and 2296 kcal/kg DM, respectively. It should be noted that these values are in the range of the mean in vivo reference values of the AMEn measurement of soybean meal of protein class 46, 48 and 50 of 2303±137, 2348±248, and 2365±178 kcal/kg DM, respectively. Thus, the energy supply provided by this microalga is comparable to that of standard-quality soybean meal.

TABLE 12
(test 14ALG069):
A: Apparent metabolizable energy and nitrogen-corrected
apparent metabolizable energy of the microalga tested
measured in 23-day-old chickens.
B: In vivo reference values measured according to the
same model for various soybean meal protein classes
(references from Adisseo, 2012).

| | Micro- | | Soybean | Soybean | Soybean |
|---|---|---|---|---|---|
| | alga | | meal | meal | meal |
| AME, | 2635.0 | Protein | 46 | 48 | 50 |
| kcal/kg | | class | | | |
| AME, | 2785.4 | | | | |
| kcal/kg | | | | | |
| DM | | | | | |
| AMEn, | 2172.0 | N | n = 15 | n = 27 | n = 80 |
| kcal/kg | | | | | |
| AMEn, | 2296.0 | AMEn, | 2303 ± 137 | 2348 ± 248 | 2365 ± 178 |
| kcal/kg | | kcal/kg | | | |
| DM | | DM | | | |

| | |
|---|---|
| A | B |

### Example 2.7

### Choice and Consumption Measurement Tests in Chicks of Feeds Comprising the Biomass According to the Invention

In each test presented below, the “microalga, strain 4, 22-h culture”, like the other raw materials, was ground on a 3-mm grid and incorporated in increasing proportions of 5%, 10% and 15% into the corn-soybean diet, while adjusting the formulation so as to have four iso-energetic, iso-protein and iso-lysine diets (Table 13), the composition of which is given in Table 14.

TABLE 13
(test 14ALG069): Apparent digestibility coefficient
of phosphorus of the microalga tested measured
in 23-day-old chickens by linear regression

| | Microalga |
|---|---|
| Digestible phosphorous (% DM)¹ | 0.86 |
| DCa phosphorous (%)² | 35.7 |

¹Confidence intervals: 0.576%-1.143%
²Calculated according to the formula: DCa = Digestible P (%)/Feed total P (%)

TABLE 14
(test 14ALG081): Formulas of the experimental feeds
used in the consumption and choice tests in chicks

| | | 5% | 10% | 15% |
|---|---|---|---|---|
| | Control | microalga | microalga | microalga |
| %, as provided | feed | feed | feed | feed |
| Corn | 52.90 | 54.28 | 55.67 | 57.06 |
| Soybean meal 48% | 35.40 | 29.50 | 23.59 | 17.69 |
| Extruded soybean | 4.00 | 4.00 | 4.00 | 4.00 |
| Soybean oil | 3.55 | 3.10 | 2.65 | 2.20 |
| Dicalcium phosphate | 1.92 | 1.78 | 1.63 | 1.49 |
| Calcium carbonate | 1.01 | 1.13 | 1.25 | 1.36 |
| Salt | 0.36 | 0.35 | 0.34 | 0.32 |
| DL-Methionine | 0.25 | 0.24 | 0.23 | 0.22 |
| L-Lysine HCl | 0.01 | 0.02 | 0.04 | 0.06 |
| Premix | 0.6 | 0.6 | 0.6 | 0.6 |
| Microalga | 0 | 5 | 10 | 15 |
| Crude protein | 22 | 22 | 22 | 22 |
| Lysine | 1.25 | 1.25 | 1.25 | 1.25 |
| Methionine | 0.58 | 0.59 | 0.60 | 0.61 |
| Met + Cys | 0.95 | 0.96 | 0.96 | 0.96 |
| Threonine | 0.86 | 0.87 | 0.88 | 0.88 |
| Dig Lysine | 1.12 | 1.12 | 1.12 | 1.12 |
| Dig Methionine | 0.57 | 0.58 | 0.58 | 0.59 |
| Dig Met + Cys | 0.85 | 0.85 | 0.85 | 0.85 |
| Dig Threonine | 0.74 | 0.75 | 0.76 | 0.77 |
| Dig Tryptophan | 0.25 | 0.24 | 0.24 | 0.24 |
| Calcium | 0.95 | 0.95 | 0.95 | 0.95 |
| Total phosphorous | 0.72 | 0.77 | 0.83 | 0.88 |
| Available | 0.40 | 0.40 | 0.40 | 0.40 |
| phosphorous | | | | |
| Metabolizable | 3000 | 3000 | 3000 | 3000 |
| energy, kcal/kg | | | | |

Two experimental tests were carried out in parallel (FIG. 2). In the first test (choice test), consumption is measured when the animal has the choice between a microalga-supplemented feed and a corn-soybean starter. In the second (consumption measurement), consumption is measured when the animal has access to only one feed, optionally supplemented with microalga.

In test 1, the consumption data at each measurement point at 7 and 9 days of age were analyzed according to a paired test procedure (XLSTAT 2010.4.02© Addinsoft 1995-2010) considering that the consumption from one feeding dish is dependent on the consumption from the other in each cage.

The consumption and weight data from test 2 were analyzed for a feed effect according to an ANOVA procedure (XLSTAT 2010.4.02© Addinsoft 1995-2010) according to the following model with a 95% confidence interval:

Yᵢ=μ+aᵢ+bᵢY=parameter

With
Y=parameter
μ=mean
aᵢ=feed effect
bᵢ=block effect

For each significant difference, the means are analyzed by a pairwise Fisher's LSD test.

1) Test 1—Choice Test

For the test with choice of feed, approximately 200 1-day-old male chicks (Ross PM3) were placed in groups of about 20 in divided metabolic cages and fed a standard starter containing wheat, corn and soybean meal. At 6 days of age the chickens are starved for 2 hours before being weighed and distributed by weight group. One hundred twenty chickens are thus selected, placed by groups of 4 into 30 divided metabolic cages and assigned at day 7 to one of the experimental treatments according to their weight (10 repetitions per treatment). Each cage contains two feeding dishes containing different feeds, corresponding to the following three experimental treatments:
Treatment 1: corn-soybean starter (feeding dish 1) and 5% microalga (feeding dish 2)
Treatment 2: corn-soybean starter (feeding dish 1) and 10% microalga (feeding dish 2)
Treatment 3: corn-soybean starter (feeding dish 1) and 15% microalga (feeding dish 2)

Feed and water are distributed ad libitum throughout the test. The experimental feeds are provided as 3.2-mm-diameter pellets. Consumption is measured at T₀₊₁ₕ, T₀₊₂ₕ, T₀₊₃ₕ, T₀₊₄ₕ and T₀₊₀ₕ at 7 and 9 days of age, with the feeding dishes in the cages being switched every hour. Between the two consumption measurements (day 8), the animals receive the wheat-corn-soybean starter.

The results show (see FIGS. 3-5) that the young animals did not reject the microalga-supplemented feeds, and this regardless of the incorporation rate. In these amounts, the microalga thus presents no palatability problem. The young chicks even tend to prefer the feeds supplemented with 5% and 10% microalga (FIG. 5) compared to conventional corn-soybean feed, which is reversed numerically when 15% microalga is added.

2) Test 2—Consumption Measurement

In a second test, the chicks have access to only one type of feed, optionally supplemented with microalga (FIG. 6). Approximately 400 1-day-old male chicks (Ross PM3) were weighed and distributed by weight group. Two hundred forty selected animals are placed by groups of 4 at day 1 in 60 undivided cages in blocks of homogeneous weight (15 blocks of 4 cages) with one feeding dish per cage, each with an experimental feed (15 repetitions per feed). In each cage, the 4 chicks are identified individually. Feed and water are distributed ad libitum throughout the test. The experimental feeds are corn-soybean starter supplemented with 0%, 5%, 10% or 15% microalga, also used in test 1 with choice of feed. Individual live weights are measured at 1, 7 and 9 days of age. Uneaten feeds are weighed and consumption per cage is measured at 7 and 9 days of age. From 0 to 7 days of age, the animals fed the feed containing 10% microalga have a significantly improved weight gain relative to the control and to the other two supplemented diets. The feed conversion ratio measured for the 10% supplemented diet is significantly improved relative to the control and to the 15% supplemented diet (1.089 vs 1.156). Furthermore, the supplementation with microalga has no effect on feed consumption when compared with the corn-soybean control (122.6 g, 125.7 g, 135.6 g and 124.6 g, respectively, for the control and the three diets supplemented 5% to 15%).

Over the total measurement period from 0 to 9 days of age (FIG. 7), the positive effect of the feed supplemented with 10% microalga on weight gain and feed conversion ratio are confirmed.

The consumption results (under non-choice conditions) show that the “microalga, strain 4, 22-h culture” tested may be included up to 15% in balanced corn-soybean feeds without affecting the performance of 0- to 9-day-old chicks.

### REFERENCES

Becker E. W. (2007) Biotechnol. Adv., 25: 207-210 Henman et al., 2012
Bourdillon A., Carré B., Conan L., Francesch M., Fuentes M., Huyghebaert G., Janssen W. M., Leclercq B., Lessire M., McNab J., Rigoni M. et Wiseman J., 1990b. Br. Poult. Sci., (31), 567-576.
Carré et Brillouet (1989, Determination of water-insoluble cell walls in feeds:

interlaboratory study. Journal Association of Official Analytical Chemists, 72,463□467).
Green S., Bertrand S., Duron M., Maillard R., 1987. Br. Poult. Sci., (28), 631-641.
Green S., Bertrand S., Duron M., Maillard R., 1987. Br. Poult. Sci., (28), 631-641.
Henman D. J. (2012) Report 4A-102, CRC Australia, 15 p
Hill F. W. et Anderson D. L., 1958. J. Nutr., (64), 587-603.
Lieve M. L. Laurens, Thomas A. Dempster, Howland D. T. Jones, Edward, J. Wolfrum, Stefanie Van Wychen, Jordan S. P. McAllister, Michelle Rencenberger, Kylea J. Parchert, and Lindsey M. Gloe. Algal Biomass Constituent Analysis: Method Uncertainties and Investigation of the Underlying Measuring Chemistries. Anal. Chem. 2012,84,1879-1887 Bourdillon et al., 1990
Prosky L., Asp N.-G., Schweitzer T. F., DeVries J. W., Furda I., 1988. Determination of total dietary fiber in foods and food products: interlaboratory study. Journal of the Association of Official Analytical Chemists, 71:1017-1023.
Skrede A., Mydland L. T., Ahlstrom Ø., Reitan K. I., Gislerod H. R., Øverland M. (2011) J. Anim. Feed Sci., 20: 131-14211
Terpstra K., De Hart N., 1974. J. Anim. Physiol. Anim. Nutr., (32), 306-320.
WO 2010/051489
WO 97/37032

1. A thraustochytrid biomass comprising at least 35% by weight of proteins relative to the weight of dry matter.

2. The biomass according to claim 1, wherein it comprises at least 45% proteins.

3. The biomass according to claim 1, wherein it comprises at least 60% proteins.

4. The biomass according to claim 1, wherein it comprises less than 20% fat by weight relative to the weight of dry matter.

5. The biomass according to claim 4, wherein it comprises less than 10% fat.

6. The biomass according to claim 1, wherein said thraustochytrids are of a genus selected from the group consisting of Aurantiochytrium, Aplanochytrium, Botryochytrium, Japonochytrium, Oblongichytrium, Parietichytrium, Schizochytrium, Sicyoidochytrium, Thraustochytrium and Ulkenia.

7. The biomass according to claim 6, wherein said thraustochytrids are selected from the species Aurantiochytrium mangrovei CCAP 4062/2; Aurantiochytrium mangrovei CCAP 4062/3; Aurantiochytrium mangrovei CCAP 4062/4; Aurantiochytrium mangrovei, CCAP 4062/5; Aurantiochytrium mangrovei CCAP 4062/6; Aurantiochytrium mangrovei CCAP 4062/1; Schizochytrium sp. 4087/3; Schizochytrium sp. CCAP 4087/1; Schizochytrium sp. CCAP 4087/4; Schizochytrium sp. CCAP 4087/5.

8. The biomass according to claim 1, wherein it has a moisture content of 1% to 95%.

9. The biomass according to claim 8, wherein it has a moisture content of 70% to 90%.

10. The biomass according to claim 8, wherein it has a moisture content of 1% to 10%.

11. A process for producing a biomass as defined in claim 1, wherein it comprises:
a. a first step of culturing thraustochytrids in a defined culture medium comprising a carbon source, a nitrogen source, a phosphorus source and salts, said first step a) being divided into two sub-steps, a first sub-step:
a1) of growth in the suitable culture medium until carbon source contents in the medium lower than 20 g/L are obtained, followed by a second sub-step:
a2) of production wherein one or more carbon source, nitrogen source and/or phosphorus source enrichment solutions are added to the culture medium, simultaneously or successively, until a culture density of at least 40 g/L dry matter is obtained;
b. a second step of recovering the biomass obtained in the first step by separating said biomass from the culture medium.

12. The process according to claim 11, wherein in step a2) the carbon source content is maintained between 0 and 50 g/L, the nitrogen source content is maintained between 0.5 and 5 g/L and the phosphorous source content is maintained between 0.5 and 5 g/L.

13. The process according to claim 11, wherein it comprises a third step:
c. of drying the biomass recovered in the second step.

14-15. (canceled)

16. A method for improving animal performance comprising administering to the animal the thraustochytrid biomass as described in claim 1.

17. The method according to claim 16, wherein the improvement in performance is evaluated by measuring consumption, weight gain or feed conversion ratio.

18. A cosmetic or pharmaceutical composition for humans or animals comprising a biomass as described in claim 1.

19. A food product, wherein it comprises a biomass as described in claim 1.

20. A livestock feed, wherein it comprises 1% to 60% of a biomass as described according to claim 1.

21. The feed according to claim 20, wherein it comprises 1% to 20% of the said biomass.

22. The feed according to claim 21, wherein it comprises 3% to 8% of the said biomass.

23. A livestock feed, wherein it comprises 1% to 40% of a biomass according to claim 1.

24. The feed according to claim 23, wherein it comprises 5% to 10% of the said biomass.

25. (canceled)
